(19) United States
(12) Patent Application Publication
(10) Pub. No.: US 2005/0106134 A1
(43) Pub. Date: May 19, 2005

(54) Pregnancy-related enzyme activity

(75) Inventors: Guiying Nie, Melbourne (AU); Lois Adrienne Salamonsen, Kew (AU); John Kerr Findlay, Hawthorn Victoria (AU)

Correspondence Address: MCKENNA LONG & ALDRIDGE LLP, 1900 K STREET, NW, WASHINGTON, DC, 20006, US

(73) Assignee: PRINCE HENRY'S INSTITUTE OF MEDICAL RESEARCH, Melbourne (AU)

(21) Appl. No.: 10/485,508

(22) PCT Filed: Jul. 31, 2002
(86) PCT No.: PCT/AU02/01020

(30) Foreign Application Priority Data
Jul. 31, 2001 (AU) PR 6730

(51) Int. Cl. A61K 38/48

(52) U.S. Cl. 424/94.63

(57) ABSTRACT

This invention relates to the role of the enzyme proprotein convertase 5/6 in pregnancy, and in particular to the detection or modulation of proprotein convertase 5/6 and its isoforms in the uterus. This enzyme is useful in the control of fertility, the monitoring of early pregnancy, and for the detection of uterine receptivity. The invention also relates to methods of screening for compounds which have the ability to modulate the activity or expression of proprotein convertase 5/6, which may be useful in regulating fertility in mammals. Novel forms of proprotein convertase 5/6 are also disclosed and claimed.

This invention relates to the role of the enzyme proprotein convertase 5/6 in pregnancy, and in particular to the detection or modulation of proprotein convertase 5/6 and its isoforms in the uterus. This enzyme is useful in the control of fertility, the monitoring of early pregnancy, and for the detection of uterine receptivity. The invention also relates to methods of screening for compounds which have the ability to modulate the activity or expression of proprotein convertase 5/6, which may be useful in regulating fertility in mammals.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

Embryo implantation, the process by which the blastocyst attaches to and implants in the uterus, leads to the establishment of an intimate relationship between the embryo and the endometrium. Implantation is one of the most important limiting factors in establishing a successful pregnancy. It is a complex process involving active interactions between the blastocyst and the uterus. The uterus must undergo dramatic morphological and physiological changes to transform itself from a non-receptive to a receptive state. This differentiation process is primarily mediated by the coordinated effects of the ovarian-hormones, which act through their intracellular receptors to regulate gene expression, and hence to influence cellular proliferation and differentiation.

While the details of the exact molecular events occurring in the uterus during this differentiation process towards receptivity are still unknown, in principle it can be predicted that a unique set of genes is up- or down-regulated in a temporally and spatially specific manner. Indeed, induction of specific genes in the uterus during the peri-implantation period, including those encoding some growth factors and cytokines, has been reported (Huet-Hudson et al, 1990; Stewart et al, 1992; Zhu et al, 1998; Robb et al, 1998; Das et al, 1999). However, given the complexity and the as-yet imprecisely defined molecular mechanisms of the process, many other molecules critical for implantation are still unidentified.

We have used the mouse as a model in a search for hitherto unrecognised molecules which are important in the early stages of implantation. In the mouse on day 4.5 of pregnancy, the uterus undergoes dramatic morphological changes in association with cell proliferation and differentiation, leading to the acquisition of a receptive state (Finn & McLaren, 1967; Abrahamsohn & Zorn, 1993). This uterine remodelling is associated with an increase in vascular permeability at implantation sites (Psychoyos, 1973).

We hypothesised that the proliferation and differentiation of endometrial cells at this time is associated with up- or down-regulation of a number of genes, many of which are still unknown (Nie et al, 1997). To identify uterine genes which are potentially critical for uterine receptivity, we used the technique of RNA differential display (DDPCR) (Liang & Pardee, 1992 & 1993), and compared the mRNA expression patterns of implantation and interimplantation sites on day 4.5 of pregnancy (Nie et al, 2000a & b).

We found that one of the genes which was differently regulated between the two sites was that encoding proprotein (prohormone) convertase 5/6 (PC 5/6), a member of a serine proteinase family which is responsible for processing of precursor proteins to their active forms by selective proteolysis. PC 5/6 has not been previously detected in the uterus, and no role for this enzyme during embryo implantation has been suggested.

A cDNA encoding PC5, as well as the complete amino acid and nucleotide sequence of human PC5, is disclosed in PCT/CA97/00535. PC5 is described as being a prorenin-processing enzyme which is overexpressed in artherosclerotic coronary arteries. Inhibition of smooth muscle cell proliferation by antisense oligonucleotides directed against PC5 is described, as well as the use of such oligonucleotides in the prevention of restenosis. PC5 RNA was also detected in the post-partum placental cotyledon and in the testes.

Miranda et al. (1996) describes the presence of two isoforms of human PC6 protease in human T cells and its role in HIV-1 gp160 processing in CD4+ T cells. Its distribution in the intestine, adrenal glands, lung, ovaries, testes, brain, spleen, kidney and liver is described. There is no mention of the presence of PC6 in the uterus.

Only the role of PC5 in the cleaving of human prorenin is discussed. There is no disclosure or suggestion in either of these documents that PC5 might be present in the uterus, or that it might play a role in fertility.

PC 5/6 has been detected in the thyroid, parathyroid, gut, adrenals, nervous system, and the cardiovascular system, but has not been reported as being present in any gonadal tissue or other reproductive organ. (Seidah & Chretien, 1999). In another paper (Rancourt & Rancourt, 1997) there is a mention of PC6 expression detected by in situ hybridisation in decidua and trophoblast cells, but no additional data is provided.

We have examined the uterine expression of PC 5/6 during early pregnancy in the mouse, and have demonstrated for the first time that this proprotein convertase is specifically upregulated in the mouse uterus at the sites of embryo attachment, and that its expression is restricted to the decidualized stromal cells. We have also identified a novel isoform of PC 5/6, which appears to be specifically expressed in implantation sites of the uterus during the embryo's implantation period. We have also detected PC 5/6 expression in human endometrium.

### SUMMARY OF THE INVENTION

Proprotein convertase 5/6 is believed to be useful in promoting the implantation of the fertilized egg in the uterus, development of the embryo and maintenance of pregnancy. Therefore modulation of the activity of this enzyme may be used to promote or to inhibit fertility. It is contemplated that the fertility-promoting methods of the invention will be particularly useful in assisted reproduction programs, including but not limited to in vitro fertilisation and gamete intrafallopian transfer methodologies. It is also contemplated that modulation of PC 5/6 activity could be used to prevent the establishment of a pregnancy, ie that this could be used as a contraceptive.

In a first aspect the invention provides a method of converting the uterus of a female mammal from a non-receptive to a receptive state, comprising the step of administering an effective amount of proprotein convertase 5/6 (PC 5/6), or an agonist thereof, to a female mammal in need of such treatment.

In a second aspect, the invention provides a method of promoting the implantation of a fertilised egg in a host uterus, comprising the step of administering a proprotein convertase 5/6, or an agonist thereof, to a female mammal in need of such treatment.

In a third aspect, the invention provides a method of detecting the fertile period in a female mammal, comprising the step of measuring the activity of or detecting the presence of proprotein convertase 5/6 in a biological sample from the mammal. The biological sample may be uterine tissue, washings from the uterine cavity, or another biological fluid, such as blood, plasma, serum or saliva.

In a fourth aspect, the invention provides a method of promoting fertility of a female mammal, comprising the step of stimulating the activity of proprotein convertase 5/6, in the uterus of a female mammal in need of such treatment. In these first, second and fourth aspects of the invention it will be clearly understood that the method of the invention may be used to treat either a female mammal who is to receive one of her own fertilised eggs, or one who is to receive a fertilised egg from a donor female.

In a fifth aspect, the invention provides a method of detecting whether the uterus of a female mammal is in a receptive state, comprising the step of detecting the presence or absence of PC 5/6, or its presence in increased amounts at a particular stage of the cycle compared with another stage, whereby the presence of or an increase in PC 5/6 means that the uterus is in a receptive state. The presence or absence of PC 5/6 may be assessed by detecting the activity of the enzyme itself, eg. using a specific antibody directed against the enzyme, or by detecting nucleic acid, encoding the enzyme, for example using PCR or in in situ hybridization.

In a sixth aspect, the invention provides a method of detecting an early pregnancy, comprising the step of detecting the presence of PC 5/6 or an increase of PC 5/6 above the level in the non-pregnant state.

In a seventh aspect, the invention provides a method of inhibiting the conversion of a non-receptive state of the uterus of a female mammal to a receptive state, comprising the step of administering an antagonist of PC 5/6 to a female mammal in need of such treatment.

In an eighth aspect, the invention provides a method of inhibiting fertility, including but not limited to inhibiting uterine receptivity and/or embryo implantation, in a female mammal, comprising the step of administering an antagonist of PC 5/6 to a female mammal in need of such treatment.

In a ninth aspect, the invention provides a method of screening for compounds which have the ability to modulate the activity of proprotein convertase 5/6, comprising the step of assessing the ability of a candidate compound to increase or decrease proprotein convertase 5/6 activity. The person skilled in the art will appreciate that a wide range of activities can be measured, for example stimulation of gene expression, stimulation of activation of PC 5/6 or inhibition of degradation by PC 5/6.

In a tenth aspect, the invention provides a method of identifying molecules necessary for implantation, comprising the step of testing a candidate molecule for the ability to promote the conversion of protein precursors cleavable by PC 5/6 into mature proteins.

In an eleventh aspect, the invention provides a nucleic acid molecule encoding an isoform of PC 5/6 which: (a) is present at the implantation sites in pregnant uterus during the implantation period but not in intestine or other tissues known to contain PC 5/6; (b) encodes an RNA transcript of about 5.5 kb; and (c) is able to hybridize under at least moderately stringent conditions to the sequence:

| | | |
|---|---|---|
| 1 | GAAGTTAGTT GTGCGCGCTG CTTAGCGCGC GAGCCAGCGG GCGGGCGAAG | SEQ ID NO: 4 |
| 51 | GCGGCGAAGC GTCGGGACCA TGGACTGGGA CTGGGGGAAC CGCTGCAGCC | |
| 101 | GCCCGGGACG GCGGGACCTG CTGTGCGTGC TGGCACTGCT CGCCGGCTGT | |
| 151 | CTGCTCCCGG TATGCCGGAC GCGCGTCTAC ACCAACCACT GGGCAGTGAA | |
| 201 | GATCGCCGGC GGCTTCGCGG AGGCAGATCG CATAGCCAGC AAGTACGGAT | |
| 251 | TCATCAACGT AGGACAGATC GGTGCACTGA AGGACTACTA TCACTTCTAC | |
| 301 | CATAGTAGGA CCATTAAAAG GTCTGTTCTC TCGAGCAGAG GAACCCACAG | |
| 351 | TTTCATTTCA ATGGAACCAA AGGTGGAGTG GATCCAACAG CAAGTGGTGA | |
| 401 | AAAAAAGAAC CAAGAGGGAT TATGACCTCA GCCATGCCCA GTCAACCTAC | |
| 451 | TTCAATGATC CCAAGTGGCC AAGTATGTGG TACATGCACT GCAGTGACAA | |
| 501 | CACCCATCCT TGCCAGTCAG ACATGAATAT CGAAGGAGCC TGGAAGAGAG | |
| 551 | GCTACACGGG GAAGAATATC GTGGTGACTA TCCTGGATGA CGGCATCGAG | |
| 601 | AGAACCCACC CAGATCTGAT GCAAAACTAC GATGCTCTGG CAAGTTGCGA | |
| 651 | TGTGAATGGG AATGACTTGG ACCCGATGCC TCGTTATGAT GCAAGCAATG | |
| 701 | AGAACAAGCA TGGGACCCGC TGTGCCGGAG AAGTGGCAGC CACTGCAAAC | |
| 751 | AACTCTCACT GCACTGTCGG GATCGCTTTC AACGCCAAGA TTGGAGGTGT | |
| 801 | GAGAATGCTG GATGGTGATG TCACTGACAT GGTGGAGGCA AAGTCTGTCA | |
| 851 | GCTACAACCC ACAGCATGTG CACATCTACA GTGCCAGCTG GGGACCAGAT | |
| 901 | GACGATGGCA AGACTGTGGA TGGGCCAGCT CCCCTCACCC GGCAAGCCTT | |
| 951 | TGAGAATGGC GTGAGAATGG GGCGGAGAGG CCTTGGATCT GTGTTTGTGT | |
| 1001 | GGGCATCTGG CAATGGTGGA CGGAGCAAGG ATCACTGTTC TTGTGATGGC | |
| 1051 | TATACCAACA GCATCTATAC CATCTCCATC AGCAGTACGG CCGAAAGTGG | |
| 1101 | AAAGAAACCT TGGTACTTGG AAGAGTGTTC ATCTACACTG GCTACAACCT | |
| 1151 | ACAGCAGTGG AGAATCCTAT GATAAGAAAA TAATCACTAC TGATCTAAGG | |
| 1201 | CAGCGATGCA CAGACAATCA CACTGGAACG TCAGCCTCAG CCCCCATGGC | |
| 1251 | TGCTGGAATC ATTGCCCTGG CCCTAGAAGC CAATCCGTTT CTGACCTGGA | |
| 1301 | GAGACGTGCA GCATGTTATT GTCAGGACTT CCCGTGCGGG ACATTTGAAC | |
| 1351 | GCTAATGACT GGAAAACCAA TGCTGCTGGT TTTAAGGTGA GCCATCTCTA | |
| 1401 | TGGATTTGGA CTGATGGATG CCGAAGCCAT GGTGATGGAA GCAGAGAAGT | |
| 1451 | GGACAACTGT TCCTCAGCAG CACGTGTGTG TGGAAAGCAC AGACCGACAA | |
| 1501 | ATCAAGACCA TTCGACCAAA CAGTGCAGTG CGCTCCATCT ACAAAGCCTC | |
| 1551 | AGGCTGCTCG GATAATCCCA ACCATCACGT CAATTACCTG GAGCATGTAG | |
| 1601 | TTGTGCGTAT TACCATCACA CACCCACGGA GGGGAGACCT GGCCATCTAT | |
| 1651 | CTGACATCAC CCTCAGGAAC CAGATCCCAG CTCTTGGCCA ACAGGCTCTT | |
| 1701 | TGATCATTCC ATGGAAGGGT TTAAGAACTG GGAGTTCATG ACTATTCATT | |
| 1751 | GCTGGGGAGA ACGGGCTGCT GGGGACTGGG TCCTCGAAGT TTATGATACG | |
| 1801 | CCATCTCAGC TGAGGAACTT CAAGACTCCA GGTAAATTGA AAGAATGGTC | |
| 1851 | CTTAGTCCTC TATGGCACGT CCGTACAGCC ATACTCCCCA ACCAACGAGT | |
| 1901 | TTCCCAAAGT GGAACGCTTC CGCTACAGCC GAGTGGAAGA CCCCACAGAT | |
| 1951 | GACTACGGTG CTGAAGATTA TGCAGGTCCC TGTGACCCTG AATGCAGTGA | |
| 2001 | GGTTGGATGT GACGGGCCAG GACCAGATCA CTGCAGTGAC TGCTTACACT | |
| 2051 | ACTACTACAA GCTGAAAAAT AACACCAGAA TCTGTGTCTC CAGCTGCCCT | |
| 2101 | CCTGGCCACT ACCATGCTGA CAAGAAGAGG TGCCGGAAGT GTGCCCCAAA | |
| 2151 | CTGCGAGTCC TGCTTTGGCA GCCATGGTGA TCAGTGCCTC TCCTGTAAAT | |
| 2201 | ATGGCTACTT CCTGAATGAA GAAACTAGCA GCTGTGTTAC TCAGTGCCCT | |
| 2251 | GATGGATCAT ACGAGGATAT CAAGAAAAAT GTCTGTGGGA AATGCAGTGA | |
| 2301 | GAACTGCAAG GCATGCATTG GATTTCACAA CTGCACAGAG TGCAAGGGCG | |
| 2351 | GGTTAAGTCT TCAGGGATCC CGCTGTTCGG TCACCTGCGA GGATGGACAG | |
| 2401 | TTCTTCAATG GTCACGACTG CCAGCCCTGC CATCGCTTCT GTGCTACTTG | |
| 2451 | CTCTGGGGCC GGAGCAGATG GATGTATTAA CTGCACCGAG GGGTATGTCA | |
| 2501 | TGGAAGAGGG AAGGTGTGTA CAAAGTTGTA GTGTGAGCTA CTACTTGGAC | |
| 2551 | CACTCTTCAG AGGGTGGCTA CAAATCCTGC AAGAGATGTG ATAACAGCTG | |
| 2601 | TTTGACATGC AATGGGCCAG GATTCAAGAA CTGTTCCAGC TGCCCCAGTG | |
| 2651 | GATATCTTTT AGACTTAGGA ACGTGTCAGA TGGGAGCCAT CTGCAAGGAT | |
| 2701 | GCTACGGAAG AGTCCTGGGC AGAAGGAGGC TTCT | |

but is not able to hybridize under at least moderately stringent conditions to the sequences:

| | | |
|---|---|---|
| 1 | TGGAAGGCAA GGACTGGAAT GAAGCCGTGC CCACTGAAAA GCCATCTTTG | SEQ ID NO: 5 |
| 51 | GTGAGGAGTC TGCTGCAGGA TCGACGAAAG TGGAAAGTTC AAATCAAAAG | |
| 101 | AGATGCAACG AGCCAGAATC AACCTTGTCA CTCTTCTTGT AAAACCTGCA | |
| 151 | ATGGATCTCT CTGCGCTTCA TGTCCCACAG GTATGTACCT GTGGCTGCAG | |
| 201 | GCCTGTGTTC CTTCCTGTCC CCAAGGCACT TGGCCATCAG TCACCAGTGG | |
| 251 | CAGCTGTGAA AAGTGTTCCG AGGACTGTGT CTCCTGCTCC GGTGCCGACC | |
| 301 | TTTGCCAACA GTGCCTGAGC CAGCCGGACA ACACTCTGCT TCTCCATGAG | |
| 351 | GGCAGGTGCT ACCACAGTTG CCCAGAG; | |
| or | | |
| 1 | AGGGAGGCTG AGTTTTACGA GCACACCAAG ACTGCTCTGC TAGTGACCTC | SEQ ID NO: 6 |
| 51 | TGGCGCCATG CTGTTGCTGC TGCTGGGGGC TGCTGCGGTC GTGTGGCGGA | |
| 101 | AGTCTCGAAG CAGACCTGTG GCAAAGGGGC GGTACGAAAA GCTGGCAGAA | |
| 151 | CCTACCGTGT CATACTCCTC CTACAGGAGC AGCTATCTTG ACGAGGACCA | |
| 201 | GGTGATTGAG TACAGGGACC GGGACTACGA TGAGGACGAT GAGGACGACA | |
| 251 | TCGTCTATAT GGGCCAAGAT GGCACTGTCT ACCGGAAGTT CAAGTATGGG | |
| 301 | CTGCTGGATG AGACGGAAGA TGATGAGTTG GAGTACGATG ATGAGAGCTA | |
| 351 | CTCTTACCAA TAAACAGAGC CCCCTCCCAT CTCAAACCCA CCACCCAC. | |

At the protein level, this 5.5 kb transcript encodes a PC 5/6 protein which has a different C-terminal sequence for those of the known isoforms of PC6A or PC6B.

The nucleic acid may be a cDNA, a genomic DNA, or an RNA and may be in the sense or the anti-sense orientation. Preferably the nucleic acid molecule is a cDNA.

Preferably the nucleic acid molecule comprises a sequence selected from the group consisting of:
(a) a cDNA molecule having the sequence set out in SEQ. ID. NO: 1 or SEQ ID NO: 2, or its human homologue (SEQ. ID. NO: 3);
(b) a nucleic acid molecule which is able to hybridise under at least moderately stringent conditions to (a); and
(c) a nucleic acid molecule which has at least 75% sequence identity to (a).

More preferably in (b) the nucleic acid molecule is able to hybridise under stringent conditions to the molecule of (a). More preferably in (c) the nucleic acid molecule has at least 80%, even more preferably at least 90% sequence identity to the molecule of (a).

In a twelfth aspect, the invention provides a protein having proprotein convertase 5/6 activity, which is encoded by a nucleic acid according to the invention.

It will be clearly understood that for the purposes of the invention different isoforms of PC 5/6 may be suitable. Preferably in all aspects of the invention, an isoform of PC 5/6 which is specifically expressed in implantation sites of the uterus during embryo implantation is used.

Defining appropriate hybridisation conditions is within the skill of the art. See e.g. Maniatis et al. DNA cloning volumes 1 and 11. Nucleic Acid Hybridisation. Briefly, “moderately stringent conditions” for hybridisation or annealing of nucleic acid molecules are those which
(1) employ moderate ionic strength and moderate temperature for washing, for example 0.3 M NaCl/0.03 M sodium citrate/0.1% sodium dodecyl sulfate (SDS) at 42(C, or
(2) employ during hybridisation a denaturing agent such as formamide, for example 40% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 40(C.

Another example is use of 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 (g/mL), 0.1% SDS, and 10% dextran sulfate at 42(C, with washes at 42(C in 0.2×SSC and 0.1% SDS.

In a thirteenth aspect, the invention provides a composition comprising a protein according to the invention, together with a pharmaceutically acceptable carrier.

Thus the invention provides a method of identifying agonists and antagonists of PC 5/6. In view of the crucial role in implantation indicated by the results reported herein, it is contemplated that antagonists of PC 5/6 will be useful as contraceptives, and that agonists of PC 5/6 will be useful as agents for promoting fertility or for supporting at least the early phases of pregnancy. It is further contemplated that suitable antagonists of PC 5/6 include, but are not limited to, antibodies and anti-sense nucleic acids. For example, an inhibitory antisense 17mer oligonucleotide is disclosed in International Patent Application No. PCT/CA97/00535 (WO98/04686) by Day et al.

As used herein, the term “receptive” refers to a state of the uterine lining which will allow an embryo to implant, whereas a “non-receptive” state refers to a state in which attachment and implantation are impaired. This can include a state known as “pre-receptive”.

The term “PC 5/6 antagonist” or “antagonist” refers to a substance which opposes or interferes with a functional activity of PC 5/6.

For the purposes of this specification it will be clearly understood that the word “comprising” means “including but not limited to”, and that the word “comprises” has a corresponding meaning.

### BRIEF DESCRIPTION OF THE FIGURES

FIG. 1A shows the results of RNA differential display analysis (DDPCR) of pregnant mouse uterus. The expression pattern of band 9 to be identified as PC 5/6 on the DDPCR gel is indicated by the arrow, showing much stronger intensities in implantation sites (Imp) compared to interimplantation sites (Inter) in three different mice: lane 1, animal 1; lane 2, animal 2 and lane 3, animal 3.

FIG. 1B shows the results of Northern blot analysis of mRNA detected by the cDNA extracted from band 0.9 of the DDPCR gel and used as a probe. Total RNA (15 μg) was isolated from implantation (Imp) and interimplantation sites (Inter) of day 4.5 pregnant mice. The top panel shows multiple bands, mainly at −2.2 kb and ˜3.5 kb positions; the lower panel shows the signal detected by the GAPDH probe on the same membrane as in the top panel.

FIG. 2 shows the results of Northern blot analysis of mRNA detected by the MUSPC6-fragB cDNA in the mouse uterus during early pregnancy and in the intestine. Total RNA (15 μg) was isolated from whole uterus of non-pregnant mice at estrus (NP) and 3.5 day pregnant (d3.5) mice, and from implantation sites (Imp) and interimplantation sites (Inter) on day (d) 4.5, 5.5, 6.5, 8.5 and 10.5 of pregnancy. On day 8.5 and 10.5, three types of tissue were sampled:
(1) the entire implantation unit containing the uterine implantation site, embryo and placenta [Imp (+)],
(2) uterine implantation site tissue without embryo and placenta [Imp(−)], and
(3) embryo and placenta sampled together (Emb+P1).

The top panel shows the multiple transcripts (2.2 kb, 3.5 kb, 5.5 kb, 6.5 kb and 10 kb) detected by this cDNA, and the lower panel shows the signal detected by the glyceraldehyde-3-phosphate dehydrogenase (GAPDH) probe on the same membrane.

FIG. 3 shows the results of Northern blot analysis of total RNA (15 μg) isolated from whole uterus of non-pregnant mice at metestrus (met), diestrus (die), proestrus (pro) and estrus (est). One typical cycle is shown. The top panel shows the signals detected by the MUSPC6-fragB cDNA; the lower panel shows that detected by the GAPDH probe on the same membrane.

FIG. 4 shows the results of Northern blot analysis of total RNA (15 μg) isolated from whole uterus of non-pregnant mice at estrus (NP), from implantation sites (Imp) and interimplantation sites (Inter) of day (d) 4.5 pregnant mice, and from whole uterus of 4.5 pseudopregnant (Pseudo preg) mice. Day 0 is taken as the day of finding the vaginal plug resulting from copulation. The top panel shows signals detected by the MUSPC6-fragB cDNA; the lower panel shows that detected by the GAPDH probe on the same membrane.

FIG. 5 shows the results of Northern blot analysis of the tissue specificity of the MUSPC6 mRNA. Total RNA (15 μg) was isolated from mouse muscle, whole brain, kidney, spleen, heart, testis, ovary, implantation site on day 5.5 of pregnancy (d5.5-Imp), intestine, lung, liver and placenta. The top panel shows the signals detected by MUSPC6-fragB cDNA, and the lower panel shows the signal detected by ribosomal 18s RNA probe on the same membrane.

FIG. 6 shows the results of Northern blot analysis performed to compare the mRNA transcript profiles between the uterus and the intestine. Total RNA (15 μg) was isolated from whole uterus of non-pregnant mice at estrus (NP), and from implantation sites (Imp) and interimplantation sites (Inter) on day (d) 4.5 and 5.5 of pregnancy. Total RNA (25 μg) was also isolated from the mouse intestine. The top panels show the mRNA patterns detected by the cDNA fragments representing the different domains of the MUSPC6 proteins, and the lower panel shows the signal detected by a GAPDH probe on the same membrane. (A) The multiple transcripts detected by MUSPC6-fragA/DA/DB/G/H cDNA; (B) The 3.5 kb transcript detected by MUSPC6-fragI; (C) The 6.5 kb transcript detected by MUSPC6-fragK/M.

FIG. 7 shows the results of Northern blot analysis of the tissue specificity of the MUSPC6B mRNA. Total RNA (15 μg) was isolated from mouse muscle, whole brain, kidney, spleen, heart, ovary, testis, liver, lung, intestine, and placenta, and from implantation sites (Imp) and interimplantation (Inter) sites on day 4.5 of pregnancy. The top panel shows the 6.5 kb transcript detected by MUSPC6-fragK/M cDNA, and the lower panel shows the signal detected by ribosomal 18s RNA probe on the same membrane.

FIG. 8 shows the results of Southern blot analysis of MUSPC6 DNA in the mouse. Genomic DNA was isolated from non-pregnant mouse uterus, and 10 μg was digested with the following four restriction enzymes: BamH1, EcoR1, HindIII and Bg1II probe with radio-labeled MUSPC6-fragH cDNA.

FIG. 9 shows the results of in situ hybridisation of MUSPC6 mRNA in a section of mouse uterus at an implantation site on day 4.5 of pregnancy. Artificially decidualized uterus was also tested.

FIG. 10 shows an MTE array (Clontech) demonstrating expression of PC6 mRNA in a range of human tissues, including the uterus, heart, gastrointestinal tract, kidney, thymus, lung, placenta, testis and ovary. The probe was a human PC6 cDNA.

FIG. 11 shows the results of Northern blot analysis of a range of human tissues using a human PC6 cDNA probe. Total RNA (10 g) was applied to each lane. The probe was a human PC6 cDNA.

FIG. 12 shows results of RT-PCR for PC6 on human endometrial samples taken across the normal menstrual cycle.

FIG. 13 shows results of RT-PCR of human PC6 in early pregnant human tissue (first trimester decidua and placenta), heart, perimenopausal ovary, post-menopausal ovary, and term placenta.

FIG. 14 shows results of real-time quantitative RT-PCR analysis of human PC6 mRNA from human endometrial cancer tissues (stages 1-3).

### DETAILED DESCRIPTION OF THE INVENTION

PC 5/6 may be used as an immunogen to generate anti-PC 5/6 antibodies. Such antibodies, which specifically bind to PC 5/6, are useful in assays for PC 5/6, such as radioimmunoassay, enzyme-linked immunoassay, or competitive-type receptor binding assay or radioreceptor assay, as well as in affinity purification techniques. The anti-PC 5/6 antibody should preferably bind PC 5/6 with an affinity of at least about 10⁶ L/mole, and more preferably at least about 10⁷ L/mole.

Polyclonal antibodies directed toward PC 5/6 are generally raised in animals by multiple subcutaneous or intraperitoneal injections of PC 5/6 and an adjuvant. If necessary, immunogenicity may be increased by conjugating PC 5/6 or a peptide fragment thereof to a carrier protein which is immunogenic in the species to be immunized, such as keyhole limpet hemocyanin, serum albumin, bovine thyroglobulin, soybean trypsin inhibitor, or pertussis toxin, using a bifunctional or derivatizing agent, for example, maleimidobenzoyl sulfosuccinimide ester (conjugation through cysteine residues), N-hydroxysuccinimide (conjugation through lysine residues), glutaraldehyde, succinic anhydride, SOCl2, or R¹N═C═NR, where R and R¹ are different alkyl groups.

It will be appreciated that a fragment or derivative of PC 5/6 which retains the ability to elicit anti-PC 5/6 antibody may alternatively be used as the immunogen.

Animals such as rabbits, sheep or mice are typically immunized with such PC 5/6-carrier protein conjugates combining 1 mg or 100 μg of conjugate (for rabbits or mice, respectively) with 3 volumes of Freund's complete adjuvant or another appropriate adjuvant known to those skilled in the art (eg. Montanide: Marcol), and injecting the solution intradermally at multiple sites. One month later the animals are boosted with 1/5th to 1/10th the original amount of conjugate in Freund's complete adjuvant (or other appropriate adjuvant) by subcutaneous injection at multiple sites. 7 to 14 days later animals are bled and the serum is assayed for anti-PC 5/6 antibody titre. Animals are boosted until the antibody titre plateaus. Preferably, the animal is boosted by injection with a conjugate of the same PC 5/6 with a different carrier protein and/or through a different cross-linking agent. Conjugates of PC 5/6 and a suitable carrier protein also can be made in recombinant cell culture as fusion proteins. Also, aggregating agents such as alum are used to enhance the immune response.

Monoclonal antibodies directed toward PC 5/6 are produced using any method which provides for the production of antibody molecules by continuous cell lines in culture. The modifier “monoclonal” indicates the character of the antibody as being-obtained from a substantially homogeneous population of antibodies, and is not to be construed as requiring production of the antibody by any particular method. Examples of suitable methods for preparing monoclonal antibodies include the original hybridoma method of Kohler et al 1975, and the human B-cell hybridoma method, Kozbor, 1984; Brodeur et al., 1987.

The monoclonal antibodies of the invention specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly et al., U.S. Pat. No. 4,816,567; Morrison et al 1984).

In a preferred embodiment, the chimeric anti-PC 5/6 antibody is a “humanized” antibody. Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain.

Humanization can be performed following methods known in the art (Jones et al., 1986; Riechmann et al., 1988; Verhoeyen et al., 1988), by substituting rodent complementarity-determining regions (CDRs) for the corresponding regions of a human antibody. Alternatively, it is now possible to produce transgenic animals (e.g., mice) which are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, for example, Jakobovits et al., 1993; Jakobovits et al., 1993; Bruggermann et al., 1993. Human antibodies can also be produced in phage-display libraries (Hoogenboom et al., 1991; Marks et al., 1991).

For diagnostic applications, anti-PC 5/6 antibodies typically will be labeled with a detectable moiety. The detectable moiety can be any one which is capable of producing, either directly or indirectly, a detectable signal. For example, the detectable moiety may be a radioisotope, such as ³H, ¹⁴C, ³²P, ³³P, ³⁵S, or ¹²⁵I, a fluorescent or chemiluminescent compound, such as fluorescein isothiocyanate, rhodamine, or luciferin; radioactive isotopic labels, such as ¹²⁵I, ³²P, ³³P, ¹⁴C, or ³H, or an enzyme, such as alkaline phosphatase, beta-galactosidase or horseradish peroxidase.

Any method known in the art for separately conjugating the antibody to the detectable moiety may be employed, including those methods described by David et al., 1974; Pain et al., 1981; Bayer et al., 1990.

The anti-PC 5/6 antibodies may be employed in any known assay method, such as competitive binding assays, direct and indirect sandwich assays, and immunoprecipitation assays (Zola, 1987).

Competitive binding assays rely on the ability of a labeled standard (e.g., PC 5/6 or an immunologically reactive portion thereof) to compete with the test sample analyte (PC 5/6) for binding with a limited amount of antibody. The amount of PC 5/6 in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex (David et al., U.S. Pat. No. 4,376,110). The second antibody may itself be labeled with a detectable moiety (direct sandwich assays) or may be measured using an anti-immunoglobulin antibody which is labeled with a detectable moiety (indirect sandwich assay). For example, one type of sandwich assay is an ELISA assay, in which case the detectable moiety is an enzyme.

Neutralizing anti-PC 5/6 antibodies are useful as antagonists of PC 5/6. The term “neutralizing anti-PC 5/6 antibody” as used herein refers to an antibody which is capable of specifically binding to PC 5/6, and which is capable of substantially inhibiting or eliminating the functional activity of PC 5/6 in vivo or in vitro. Typically a neutralizing antibody will inhibit the functional activity of PC 5/6 by at least about 50%, and preferably greater than 80%, as determined, for example, by an in vitro receptor binding assay.

PC 5/6 is believed to be useful in promoting the implantation of the fertilized egg, development of the placenta and the embryo, and maintenance of pregnancy. Accordingly, PC 5/6 may be utilized in methods for the diagnosis and/or treatment of a variety of fertility-related conditions, including infertility due to luteal phase defect, infertility due to failure of implantation, pre-eclampsia, early abortion, intrauterine growth restriction (IUGR), abnormal uterine bleeding, endometriosis, and early parturition, and it may provide a potential target for contraception. It is also contemplated that it may be implicated in other conditions, such as cancers, and in particular cancers of the reproductive system, such as endometrial cancer.

PC 5/6 may be formulated with other ingredients such as carriers and/or adjuvants, e.g. albumin, nonionic surfactants and other emulsifiers. There are no limitations on the nature of such other ingredients, except that they must be pharmaceutically acceptable, efficacious for their intended administration, and cannot degrade the activity of the active ingredients of the compositions. Suitable adjuvants include collagen or hyaluronic acid preparations, fibronectin, factor XIII, or other proteins or substances designed to stabilize or otherwise enhance the active therapeutic ingredient(s).

Animals or humans may be treated in accordance with this invention. It is possible but not preferred to treat an animal of one species with PC 5/6 of another species.

A number of inhibitors of proprotein convertases have been described. These include alpha₁-antitrypsin Portland (Jean et al, 1998) and N-terminal prosegments of the PCs which inhibit the parent enzyme (PC1/3, Boudreault et al 1998; Furin and PC7, Zhong et al, 1999). Although these are either non-specific or not shown for PC 5/6, similar inhibitors could be prepared which would be useful for the applications described herein.

PC 5/6 and PC 5/6 antagonists to be used for in vivo administration must be sterile. This is readily accomplished by filtration of a solution of PC 5/6 or anti-PC 5/6 antibody through sterile filtration membranes. Thereafter, the filtered solution may be placed into a container having a sterile access port, for example, an intravenous solution bag or vial having a stopper pierceable by a hypodermic injection needle. The filtered solution also may be lyophilized to produce sterile PC 5/6 or anti-PC 5/6 antibody in a powder form.

Methods for administering PC 5/6 and PC 5/6 antagonists, such as synthetic inhibitors, in vivo include injection or infusion by intravenous, intraperitoneal, intracerebral, intrathecal, intramuscular, intraocular, intraarterial, intravaginal, intrauterine, intracervical, or intralesional routes, and by means of sustained-release formulations, including suppositories.

Sustained-release formulations generally consist of PC 5/6 or PC 5/6 antagonists and a matrix from which the PC 5/6 or PC 5/6 antagonists are released over some period of time. Suitable matrices include semipermeable polymer matrices in the form of shaped articles, for example, membranes, fibers, or microcapsules. Sustained release matrices may comprise polyesters, hydrogels, polylactides, U.S. Pat. No. 3,773,919, copolymers of L-glutamic acid and gamma ethyl-L-glutamate, Sidman et al., 1983, poly(2-hydroxyethyl-methacrylate), or ethylene vinyl acetate, Langer et al., 1981 & 1982. In one embodiment of the invention, the therapeutic formulation comprises PC 5/6 or a PC 5/6 antagonist entrapped within or complexed with liposomes. For example, PC 5/6 covalently joined to a glycophosphatidyl-inositol moiety may be used to form a liposome comprising PC 5/6.

An effective amount of PC 5/6 or PC 5/6 antagonist, e.g., anti-PC 5/6 antibody, to be employed therapeutically will depend upon the therapeutic objectives, the route of administration, and the condition of the patient. Accordingly, it will be necessary for the therapist to titrate the dosage and modify the route of administration as required to obtain the optimal therapeutic effect. A typical daily dosage might range from about 1 μg/kg to up to 100 mg/kg or more, depending on the factors mentioned above. Much lower doses may be possible if the agent is administered locally. Where possible, it is desirable to determine appropriate dosage ranges first in vitro, for example, using assays for serine protease activity or specific PC 5/6 activity which are known in the art, and then in suitable animal models, from which dosage ranges for human patients may be extrapolated.

For example, the dose of a protein PC 5/6 antagonist, particularly an antibody, can be about 0.1 mg to about 500 mg, typically about 1.0 mg to about 300 mg, more typically about 25 mg to about 100 mg. The administration frequency can be appropriately selected depending upon the condition to be treated and the dosage form for the desired therapeutic effects.

The mammal may be a human, or may be a domestic or companion animal. While it is particularly contemplated that the compounds of the invention are suitable for use in medical treatment of humans, they are also applicable to veterinary treatment, including treatment of companion animals such as dogs and cats, and domestic animals such as horses, cattle and sheep, or zoo animals or feral mammals such as non-human primates, felids, canids, bovids, and ungulates.

Methods and pharmaceutical carriers for preparation of pharmaceutical compositions are well known in the art, as set out in textbooks such as Remington's Pharmaceutical selected with the planned manner of administration in mind.

The compounds of the invention may be administered orally, topically, or parenterally in dosage unit formulations containing conventional non-toxic pharmaceutically acceptable carriers, adjuvants, and vehicles. The term parenteral as used herein includes subcutaneous, intravenous, intramuscular, intrathecal, intracranial, injection or infusion techniques.

The invention also provides suitable topical, oral, aerosol, and parenteral pharmaceutical formulations for use in the novel methods of treatment of the present invention. The compounds of the invention may be administered orally as tablets, aqueous or oily suspensions, lozenges, troches, powders, granules, emulsions, capsules, syrups or elixirs. The composition for oral use may contain one or more agents selected from the group of sweetening agents, flavouring agents, colouring agents and preserving agents in order to produce pharmaceutically elegant and palatable preparations. The tablets contain the active ingredient in admixture with non-toxic pharmaceutically acceptable excipients which are suitable for the manufacture of tablets.

These excipients may be, for example, inert diluents, such as calcium carbonate, lactose, calcium phosphate or sodium phosphate; granulating and disintegrating agents, such as corn starch or alginic acid; binding agents, such as starch, gelatin or acacia; or lubricating agents, such as magnesium stearate, stearic acid or talc. The tablets may be uncoated, or may be coated by known techniques to delay disintegration and absorption in the gastrointestinal tract and thereby provide a sustained action over a longer period. For example, a time-delay material such as glyceryl monostearate or glyceryl distearate may be employed. Coating may also be performed using techniques described in the U.S. Pat. Nos. 4,256,108; 4,160,452; and 4,265,874 to form osmotic therapeutic tablets for controlled release.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers such as those based on Ringer's dextrose, and the like. Preservatives and other additives may also be present, such as anti-microbials, anti-oxidants, chelating agents, growth factors and inert gases and the like.

Generally, the terms “treating”, “treatment” and the like are used herein to mean affecting a subject, tissue or cell to obtain a desired pharmacological and/or physiological effect. The effect may be prophylactic in terms of completely or partially preventing a disease or sign or symptom thereof, and/or may be therapeutic in terms of a partial or complete cure of a disease. “Treating” as used herein covers any treatment of, or prevention of disease in a vertebrate, a mammal, particularly a human, and includes: preventing the disease from occurring in a subject who may be predisposed to the disease, but has not yet been diagnosed as having it; inhibiting the disease, ie., arresting its development; or relieving or ameliorating the effects of the disease, ie., causing regression of the effects of the disease.

The invention includes various pharmaceutical compositions useful for ameliorating disease. The pharmaceutical compositions according to one embodiment of the invention are prepared by bringing a compound of formula I, analogue, derivatives or salts thereof and one or more pharmaceutically-active agents or combinations of compound of formula I and one or more pharmaceutically-active agents into a form suitable for administration to a subject using carriers, excipients and additives or auxiliaries.

Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 20th ed. Williams & Wilkins (2000) and The British National Formulary 43rd ed. (British Medical Association and Royal Pharmaceutical Society of Great Britain, 2002; http://bnf.rhn.net), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the condition and the weight and general state of the subject. Typically, dosages used in vitro may provide useful guidance in the amounts useful for in situ administration of the pharmaceutical composition, and animal models may be used to determine effective dosages for treatment of the cytotoxic side effects. Various considerations are described, eg., in Langer, Science, 249: 1527, (1990). Formulations for oral use may be in the form of hard gelatin capsules, in which the active ingredient is mixed with an inert solid diluent, for example, calcium carbonate, calcium phosphate or kaolin. They may also be in the form of soft gelatin capsules, in which the active ingredient is mixed with water or an oil medium, such as peanut oil, liquid paraffin or olive oil.

Aqueous suspensions normally contain the active materials in admixture with excipients suitable for the manufacture of aqueous suspension. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be (a) a naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleaginous suspension. This suspension may be formulated according to known methods using suitable dispersing or wetting agents and suspending agents such as those mentioned above. The sterile injectable preparation may also be a sterile injectable solution or suspension in a non-toxic parenterally-acceptable diluent or solvent, for example, as a solution in 1,3-butanediol. Among the acceptable vehicles and solvents which may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid may be used in the preparation of injectables.

The compounds of the invention may additionally be combined with other compounds to provide an operative combination. It is intended to include any chemically compatible combination of pharmaceutically-active agents, as long as the combination does not eliminate the activity of the protein of this invention.

The invention will now be described in detail by way of reference only to the following non-limiting examples and drawings.

Materials and Methods

Animals and Tissue Preparation

Swiss outbred mice were housed and handled according to the Monash University animal ethics guidelines on the care and use of laboratory animals. All experimentation was approved by the Institutional Animal Ethics Committee at the Monash Medical Centre. Adult female mice (6-8 weeks old) were mated with fertile males of the same strain to produce normal pregnant animals, or mated with vasectomized males to produce pseudopregnant mice. The morning of finding a vaginal plug was designated as day 0 of pregnancy. Uterine tissues were collected from non-pregnant mice, or from pregnant mice on days 3-11. A selection of other mouse organs was also collected from non-pregnant mice. Tissues were snap-frozen in liquid nitrogen for Northern analysis, or fixed in 4% buffered formalin (pH 7.6) for in situ hybridisation.

For non-pregnant and day 3.5 pregnant mice, the entire uterus was collected. For day 4.5 pregnant mice, implantation sites were visualised by intravenous injections of a Chicago Blue dye solution (1% in saline, 0.1 ml/mouse) into the tail vein 5 min before killing the animals; implantation sites were separated from interimplantation sites, and both sites were retained. For pregnant mice on day 5.5 onwards, implantation and interimplantation sites were visualized without dye injection.

For non-pregnant mice, the uterus was also collected from different stages of the estrous cycle: metestrus, diestrus, proestrus and estrus. The stages of the cycle were determined by analysis of vaginal smears (Rugh, 1994). For ovarian hormone treatments, the animals were first ovariectomized under anaesthesia with avertin, without regard to the stage of the estrous cycle (Rugh, 1994).

### EXAMPLE 1

### RNA Differential Display (DDPCR) and Isolation of cDNAs

DDPCR was performed as previously described (Nie et al, 2000b), and as described originally by Liang and Pardee (1992 & 1993).

Total RNA was extracted from pools of implantation or interimplantation site tissues of mice by acid guanidinium thiocyanate-phenol-chloloroform extraction (Chomczynski and Sacchi, 1987). The RNA was then treated with RNase-free DNase in the presence of placental RNase inhibitor to remove any contaminating DNA. The amount of RNA in the final preparation was determined spectrophotometrically, and the RNA quality was evaluated by the ratio of OD260 over OD280 (>1.8). DNA-free RNA (1(g) from the implantation and interimplantation sites of three animals was used as the template for the first-strand cDNA synthesis in a 20 μl reaction mixture in the presence of 20 μM dNTPs, 50 μM oligo-dT anchored primers (one of T12MG, T12MC, T12MA and T12MT), as shown in Table 1, 10 mM DTT, 10 U RNasin ribonuclease inhibitor (Promega, Madison, Wis.), 25 U AMV reverse transcriptase (Boehringer Mannheim, Nunawading, Victoria, Australia), and the cDNA synthesis buffer (Boehringer Mannheim). The synthesised cDNA was then amplified by PCR in 20 μl with the following components: 2 μl of cDNA, 1×PCR buffer (10 mM Tris-HCl, 1.5 mM MgCl₂, 50 mM KCl, pH 8.3), 10 μM dNTPs, 10 picomoles of one random decamer (Table 1, Operon 10-mer kit A; Operon, Alameda, Calif.), 50 picomoles of one oligo-dT anchored primer (as used in cDNA synthesis), 2 (Ci of ³³P-dATP (Du Pont Ltd., North Sydney, Australia) and 1 U of Taq DNA polymerase (Boehringer Mannheim). The PCR was performed in a Hybaid OmniGene PCR system (Hybaid Ltd, Teddington, Middlesex, UK) with the following conditions: initial denaturation at 94° C. for 5 min; then 40 cycles of denaturation at 94° C. for 30 sec, primer annealing at 39° C. for 2 min, and a final extension at 72° C. for 30 sec; and a final extension at 72° C. for 10 min. The subsequent PCR products (4 μl) were run on 6% high-resolution polyacrylamide/urea gel (Liang & Pardee, 1992) and visualised by autoradiography. The majority of amplified fragments were shared between the implantation and interimplantation sites. Bands unique to implantation sites compared to interimplantation sites were identified as of interest. The 80 PCR primer combinations (20 random 10 mers combined with 4 oligo-dT anchored primers) used in the initial DDPCR analysis are shown in Table 1.

The candidate bands obtained from the DDPCR analysis were excised from the dried sequencing gel, incubated with 100 μl H₂O and 2 drops of oil at 100° C. for 15 min, followed by room temperature incubation for 1 hr, and then the mixture was vigorously vortexed before centrifugation for 5 min to remove debris and obtain the cDNA. The eluted cDNAs were reamplified by PCR using the conditions described above. The differential display pattern was further confirmed by Northern blotting analysis using as probes the reamplified PCR products, which were generated by random hexamer labelling.

TABLE 1

The 80 (4 × 20) primer combinations used
in DDPCR
3′ primers: Oligo-(dT) anchored primers,
custom-made

| | | | |
|---|---|---|---|
| Primer | Code | Sequence | SEQ ID NO. |

| | | | |
|---|---|---|---|
| 1 | T12MA | TTTTTTTTTTTT(G,A,C)A | 7 |
| 2 | T12MC | TTTTTTTTTTTT(G,A,C)C | 8 |
| 3 | T12MG | TTTTTTTTTTTT(G,A,C)G | 9 |
| 4 | T12MT | TTTTTTTTTTTT(G,A,C)T | 10 |

5′ Primers: 10 mers, from Operon

| | | | |
|---|---|---|---|
| | | | SEQ |
| Prim- | | | ID |
| er | Code | Sequence | NO. |

| | | | |
|---|---|---|---|
| 1 | OPA-01 | CAGGCCCTTC | 11 |
| 2 | OPA-02 | TGCCGAGCTG | 12 |
| 3 | OPA-03 | AGTCAGCCAC | 13 |
| 4 | OPA-04 | AATCGGGCTG | 14 |
| 5 | OPA-05 | AGGGGTCTTG | 15 |
| 6 | OPA-06 | GGTCCCTGAC | 16 |
| 7 | OPA-07 | GAAACGGGTG | 17 |
| 8 | OPA-08 | GTGACGTAGG | 18 |
| 9 | OPA-09 | GGGTAACGCC | 19 |
| 10 | OPA-10 | GTGATCGCAG | 20 |
| 11 | OPA-11 | CAATCGCCGT | 21 |
| 12 | OPA-12 | TCGGCGATAG | 22 |
| 13 | OPA-13 | CAGCACCCAC | 23 |
| 14 | OPA-14 | TCTGTGCTGG | 24 |
| 15 | OPA-15 | TTCCGAACCC | 25 |
| 16 | OPA-16 | AGCCAGCGAA | 26 |
| 17 | OPA-17 | GACCGCTTGT | 27 |
| 18 | OPA-18 | AGGTGACCGT | 28 |
| 19 | OPA-19 | CAAACGTCGG | 29 |
| 20 | OPA-20 | GTTGCGATCC | 30 |

To avoid embryonic contamination, the embryos were removed under the light microscope from the implantation sites. After the DDPCR analysis, the differential display pattern was further verified by the Northern blotting analysis and cDNAs from those confirmed bands were sub-cloned into the pGEM-T vector (Promega, Madison, Wis., USA) and sequenced manually.

For Northern blot analysis, no attempt was made to separate the embryos from the decidua before day 8 of pregnancy, but for 8- and 11-day pregnant mice, embryos were separated from the uterine tissue. Total RNA was extracted from whole uteri or from pools of implantation or inter-implantation sites by the acid guanidinium thiocyanate-phenol-chloroform extraction (GTC) method (Chomczynski & Sacchi, 1987). RNA (10-15 (g) was denatured at 50° C. for 60 min in 50% dimethylsulfoxide (DMSO) and 1M glyoxal, and the denatured RNA was fractionated by electrophoresis through a 1.2% agarose gel in 10 mM sodium phosphate buffer (pH 7.0) and transferred to positively charged nylon membranes (Hybond-N+, Amersham) by overnight capillary blotting in 5×SSPE (1×SSPE=150 mM NaCl, 10 mM NaH2PO4, 1 mM EDTA, pH 7.4). Membranes were baked at 80° C. for 2 h followed by 3 min UV cross linking.

Transcript size was estimated by comparison with RNA size standards (Gibco-BRL, Gaithersburg, Md. USA). A simplified filter paper sandwich blotting method (Jones & Jones, 1992; Nie et al, 2000b) was used for the hybridisation process at 42° C. overnight, without a prehybridisation step. The hybridisation buffer contained 50% formamide, 6×SSPE (900 mM sodium chloride, 60 mM sodium phosphate, pH 7.4, 6 mM EDTA), 5× Denhardt's solution (0.1% Ficoll, 0.1% polyvinylpyrrolidone, 0.1% bovine serum albumin), 0.5% SDS, 0.1 mg/ml sheared herring sperm DNA, and ³²P-dCTP-labelled probes. The radio-labeled cDNA probes were generated by random primer labeling of 25 ng cDNA with [³²P]deoxy-CTP (50 (Ci/reaction).

Unincorporated nucleotides were removed with a MicroSpin S-200 HR column (AMRAD Pharmacia Biotech, Melbourne, Australia). Following hybridisation, the blots were rinsed twice with 5×SSPE at 37° C., then twice for 15 min each at 37° C. with 2×SSC/0.1% SDS (w/v) (1×SSC=150 mM NaCl, 15 mM Na₃ citrate, pH 7.4). In some cases, additional washes were also performed with 0.5 or 1×SSC/0.1% SDS for 15 min at 60° C. To determine lane to lane loading variation, each blot was also probed with a mouse cDNA probe for glyceraldehyde-3-phosphate dehydrogenase (GAPDH) or 18S ribosomal RNA. Between hybridisations, blots were stripped by incubation at 80° C. for 3 h in 1 mM EDTA/0.1% SDS followed by rinsing in H₂O.

For reverse transcriptase-polymerase chain reaction (RT-PCR), 1 μg DNA-free total RNA was reverse-transcribed at 46° C. for 1-1.5 h in 20 μl reaction mixture, using 100 ng random hexanucleotide primers and AMV reverse transcriptase (Boehringer-Mannheim, Nunawading, Vic., Australia) with the cDNA synthesis buffer. The PCR was performed in a total volume of 40 μl with 1-1.5 μl of the RT reaction, 1×PCR buffer, 20 μM dNTPs, 10 pmol forward and reverse primers and 2.5 units of Taq DNA polymerase (Boehringer-Mannheim), in 3 stages as follows:
(a) one cycle of an incubation for 5 min at 95° C., 1 min at 52° C.-60° C., and 2 min at 72° C.;
(b) 32 cycles with a denaturation for 45 sec at 95° C., annealing at 52° C.-60° C. for 50 sec and extension at 72° C. for 1 min; and
(c) incubation for 5 min at 72° C.

The PCR products were subjected to electrophoresis (100 v/0.5×TBE buffer) on 1.5% agarose gel, and stained with ethidium bromide. Bands of interest were cut out from the agarose gels, purified with the Qiaquick gel extraction kit (Qiagen Pty Ltd., Clifton Hill, Vic., Australia), cloned into a pGEM-T easy vector (Promega) according to the manufacturer's instructions and sequenced on an automated sequencer (Applied Biosystems, ABI Prism™, 377 DNA Sequencer) using the ABI Prism BigDye terminator cycle sequencing ready reaction kit.

The primer sequences used to amplify the following fragments of mouse proprotein convertase PC6 (MUSPC6 GenBank Accession No.: D12619; 2994 bp)) from mouse uterine RNA were:

(1) MUSPC6-fragA (171 bp), upper primer

| | |
|---|---|
| 5′ GTT AGT TGT GCG CGC TGC TTA 3′ | SEQ ID NO: 31 |
| [nucleotide (nt) 4-24] | |

and lower primer

| | |
|---|---|
| 5′ GCG CGT CCG GCA TAC C 3′ | SEQ ID NO: 32 |
| (nt 159-174) | |

(2) MUSPC6-fragB (422 bp), upper primer

| | |
|---|---|
| 5′ CAC CCA TCC TTG CCA GTC AG 3′ | SEQ ID NO: 33 |
| (nt 501-520) | |

and lower primer

| | |
|---|---|
| 5′ CAT CCA CAG TCT TGC CAT CGT 3′ | SEQ ID NO: 34 |
| (nt 902-922). | |

(3) MUSPC6-fragD (656 bp), upper primer

| | |
|---|---|
| 5′ GAT CGG TGC ACT GAA GGA CTA 3′ | SEQ ID NO: 35 |
| (nt 267-287) | |

and lower primer
5′ CAT CCA CAG TCT TGC CAT CGT 3′ (nt 902-922);
this fragment was digested with HindIII, and two smaller fragments (177 bp and 479 bp) were obtained; the 177 bp was then designated as MUSPC6-fragDA and the 479 bp as MUSPC6-fragDB.

MUSPC6-fragG (653 bp), upper primer

| 5′ CTG GCA GCA TGT TAT TGT CA 3′ | SEQ ID NO: 36 |
|---|---|
| (1305-1324) | |

and lower primer

| 5′ CGT AGT CAT CTG TGG GGT CTT 3′ | SEQ ID NO: 37 |
|---|---|
| (1937-1957). | |

(5) MUSPC6-fragH (672 bp), upper primer

| | |
|---|---|
| 5′ CCA CTA CCA TGC TGA CAA GA 3′ | SEQ ID NO: 38 |
| (nt 2106-2125) | |

and lower primer

| | |
|---|---|
| 5′ AGA ACT TTT CGC TGA CAG AGA 3′ | SEQ ID NO: 39 |
| (nt 2757-2777) | |

(6) MUSPC6-fragI (227 bp), upper primer

| | |
|---|---|
| 5′ GTA TGC TTG TGA AAA AGA ACA 3′ | SEQ ID NO: 40 |
| (nt 2735-2755) | |

and lower primer

| | |
|---|---|
| 5′ CCC TGC ACA AAC TTG AGA TAG 3′ | SEQ ID NO: 41 |
| (nt 2941-2961) | |

The following two fragments were cloned by RT-PCR from mouse intestine RNA on the basis of the MUSPC6B cDNA sequence (Accession: D17583, 5208 bp):

(1) MUSPC6-fragK (377 bp), upper primer

| | |
|---|---|
| 5′ TGG AAG GCA AGG ACT GGA ATG 3′ | SEQ ID NO: 42 |
| (nt 2522-2542) | |

and lower primer

| | |
|---|---|
| 5′ CTC TGG GCA ACT TGT GTA GCA 3′ | SEQ ID NO: 43 |
| (nt 2876-2898) | |

(2) MUSPC6-fragM (398 bp), upper primer

| | |
|---|---|
| 5′ AGG GAG GCT GAG TTT TAC GAG 3′ | SEQ ID NO: 44 |
| (nt 4285-4305) | |

and lower primer

| | |
|---|---|
| 5′ GTG GGT GGT GGG TTT GAG 3′ | SEQ ID NO: 45 |
| (nt 4665-4682). | |

### EXAMPLE 2

### Analysis of MUSPC6 Gene by Southern Blotting

Total genomic DNA was isolated from non-pregnant uterus and from kidney, using the DNeasy Tissue Kit (Qiagen Pty Ltd). A total amount of 10 μg was then digested separately with an excess of several restriction endonucleases (BamH1, EcoR1, HindIII, and BglII) at 37° C. for 14 hours, and fractionated on 0.8% agarose gel. The DNA was then blotted on to positively charged nylon membranes (Hybond-N, Amersham) using the standard Southern blotting procedure (Sambrook et al. 1989) and probed with radio-labeled cDNA, as described for the Northern analysis.

### EXAMPLE 3

### Artificial Decidualization of Mouse Uterus and In Situ Hybridisation

To induce artificial decidualization in the mouse uterus, non-pregnant mice were ovariectomised and treated with estradiol and progesterone as described by Finn and Pope (Finn and Pope. 1994), and uteri collected 48 hours after the administration of oil stimulus.

Sense and anti-sense digoxygenin (DIG)-labelled RNA probes against PC 5/6 (clone 9.5) were generated using the DIG RNA Labeling kit (Boehringer Mannheim), and the concentrations determined according to the manufacturer's instructions. Five micron sections of formalin-fixed paraffin-embedded tissues were subjected to in situ hybridisation as described by (Komminoth, 1992). All sections were counterstained with Mayer's hematoxylin.

### EXAMPLE 4

### DDPCR Analysis and Confirmation of Clone 9.5 by Northern Blotting

To identify genes which are potentially critical for the initial process of embryo implantation in the mouse, we compared the gene expression pattern of implantation and interimplantation sites in the uterus on day 4.5 of pregnancy, using the DDPCR technique. Seventeen bands, of which the intensities were different between the two sites, were detected on DDPCR gels (Nie et al., 2000 One of these bands, band 9, was fully analysed.

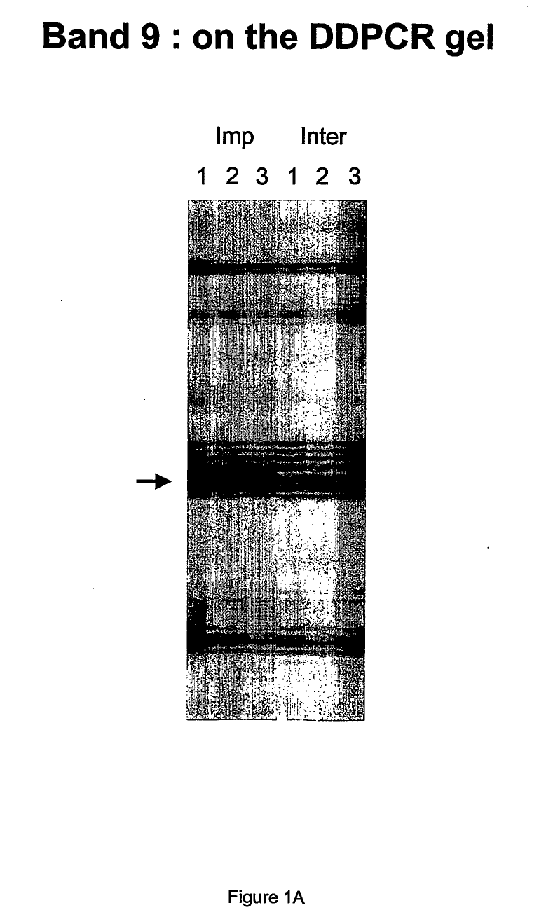

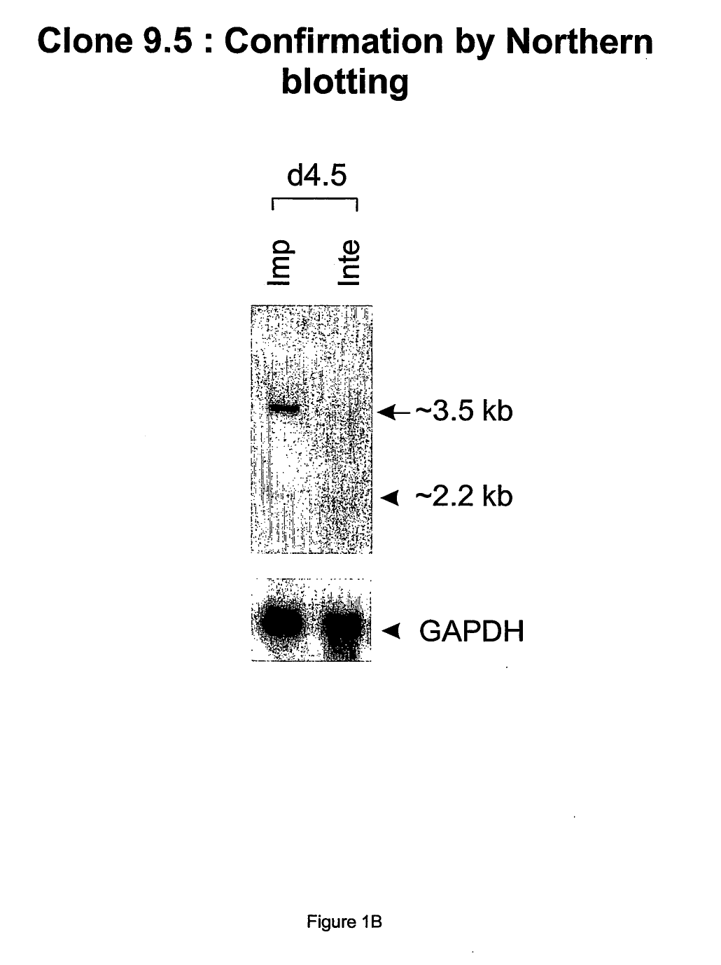

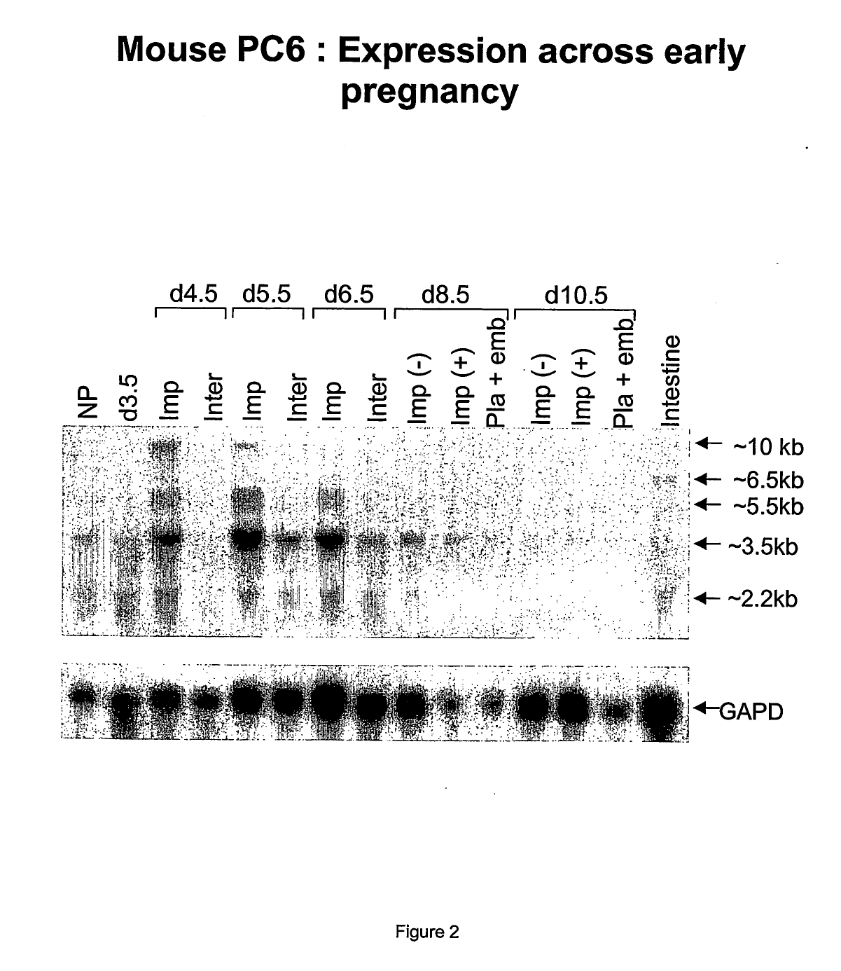

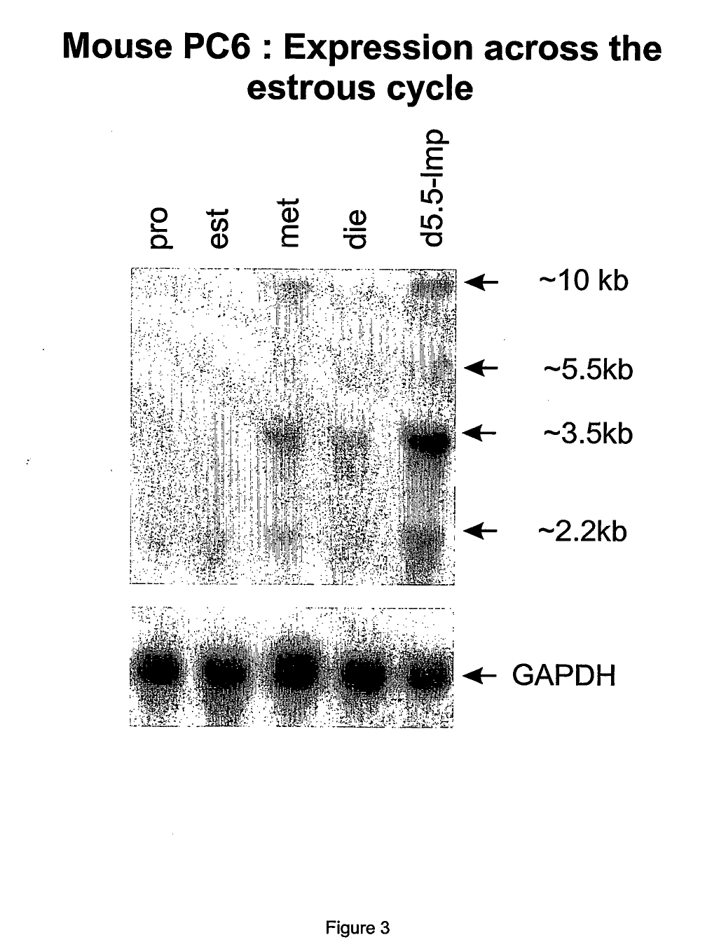

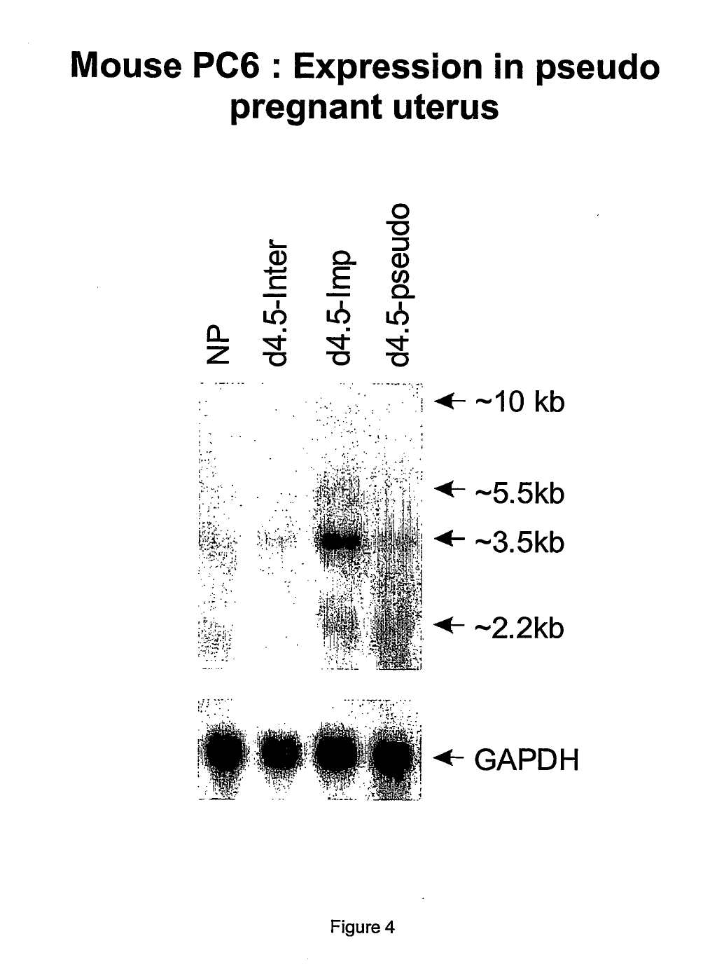

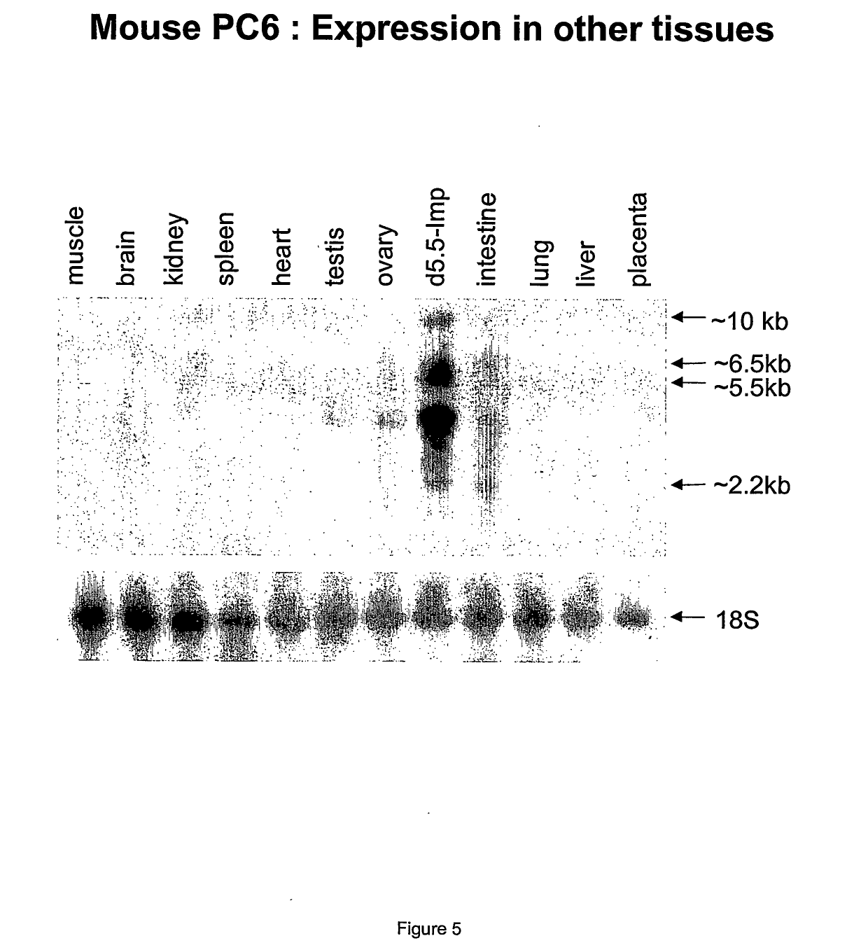

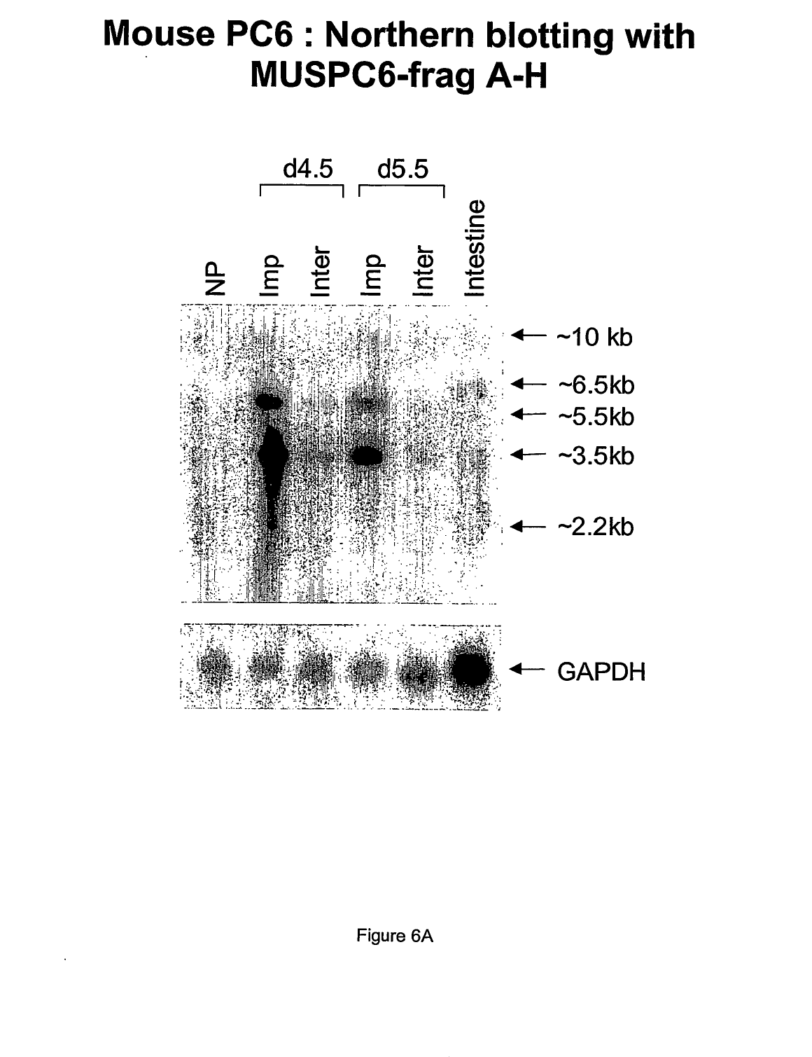

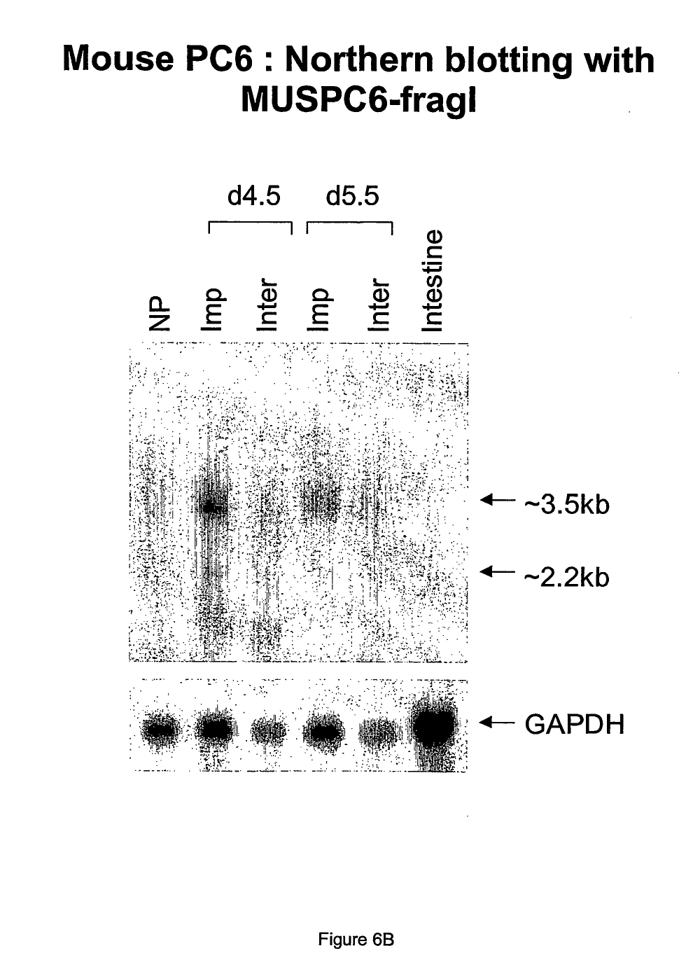

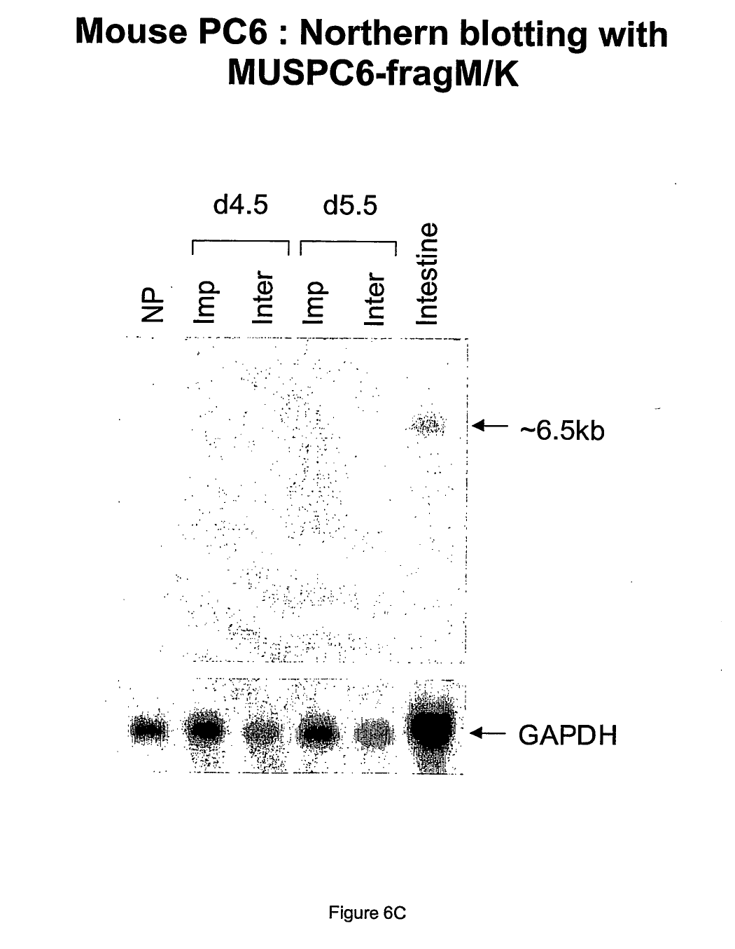

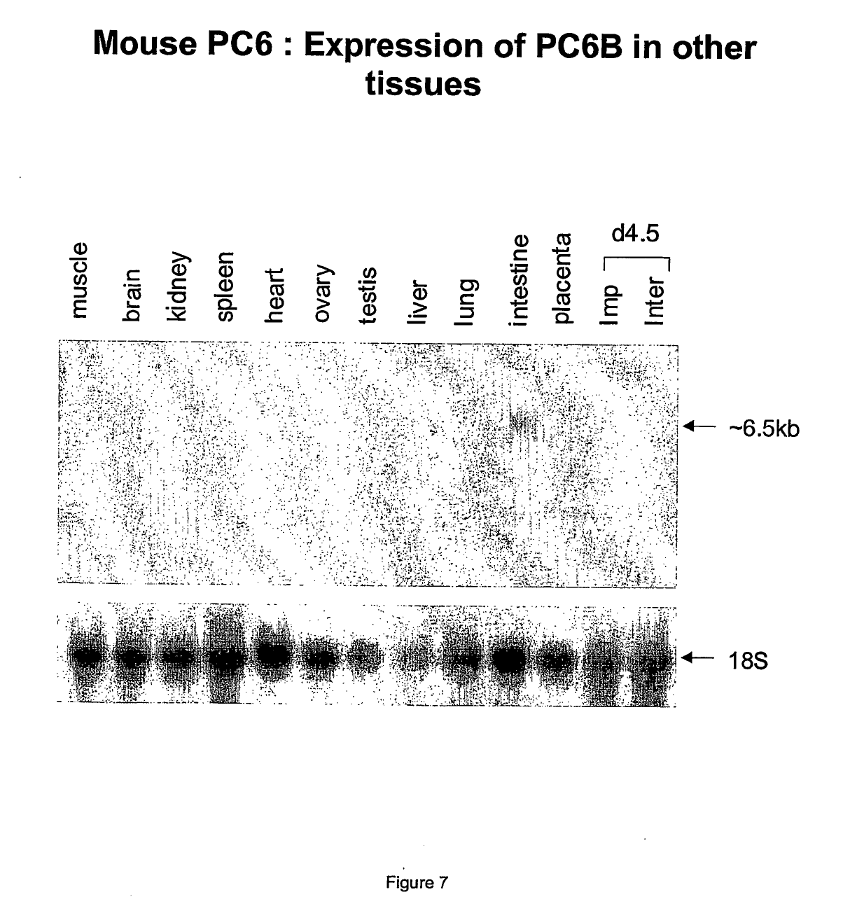

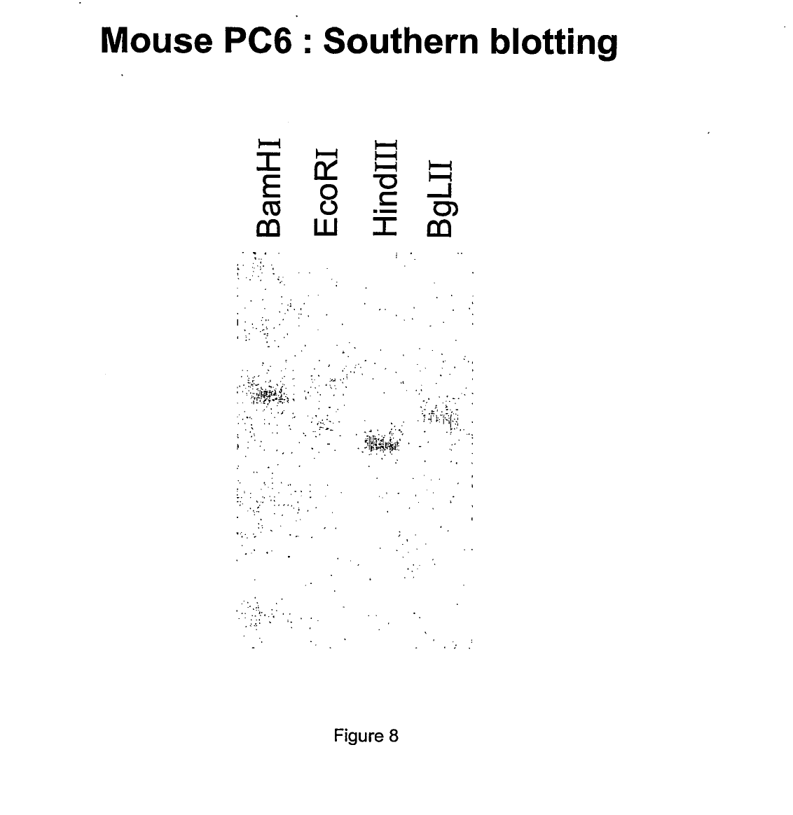

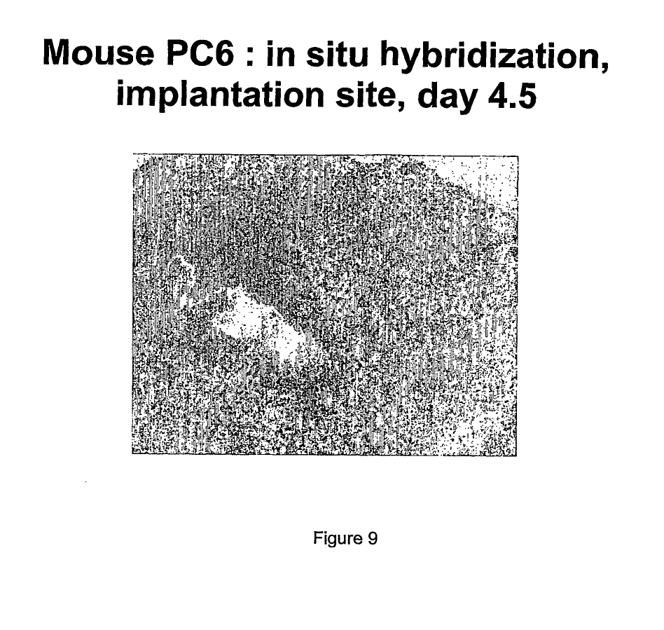

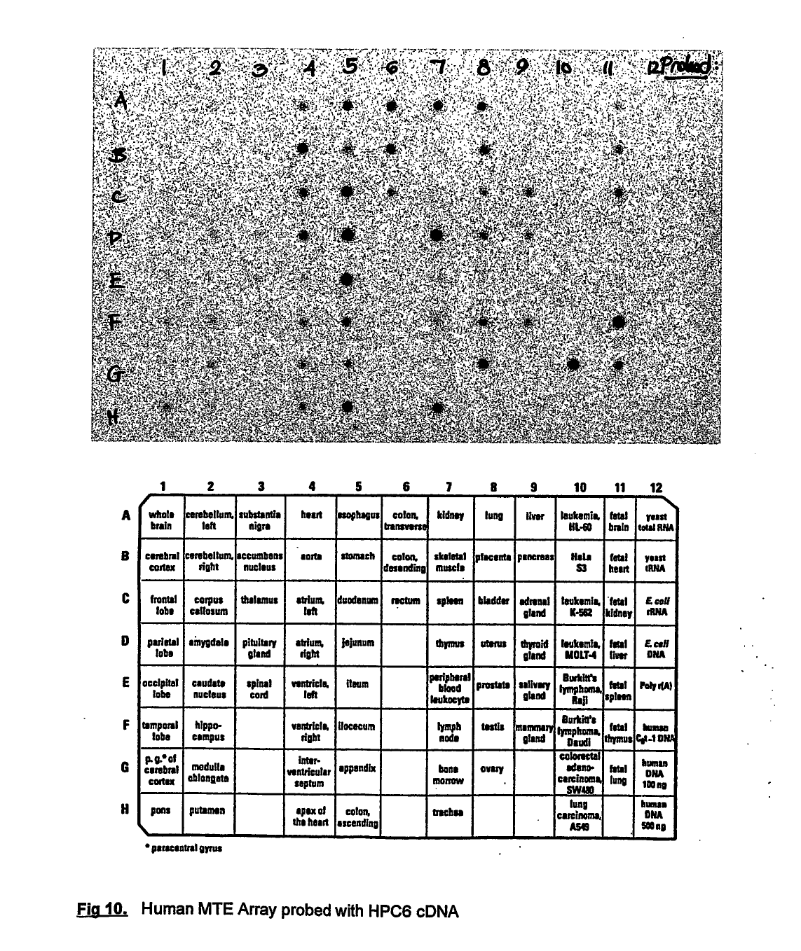

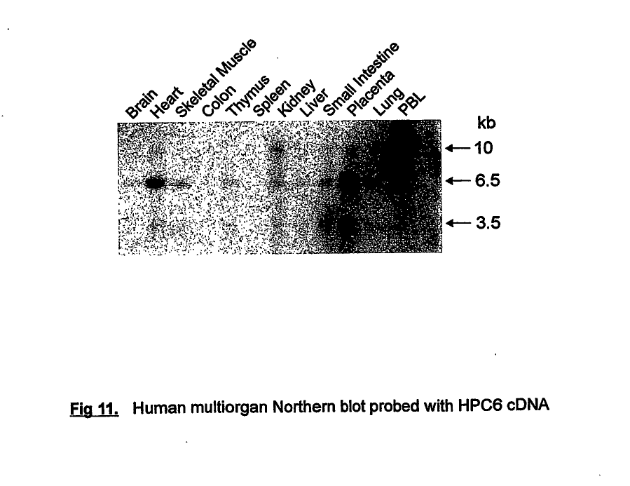

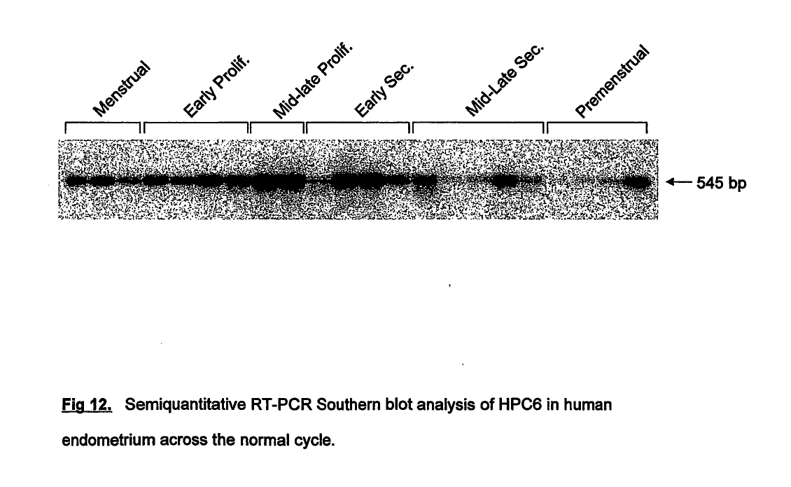

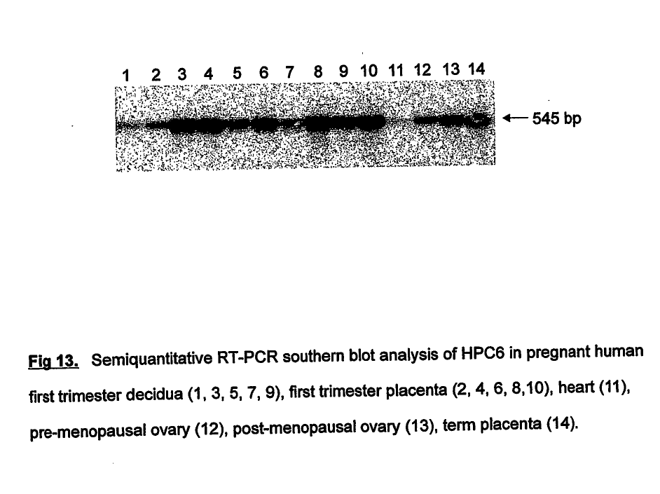

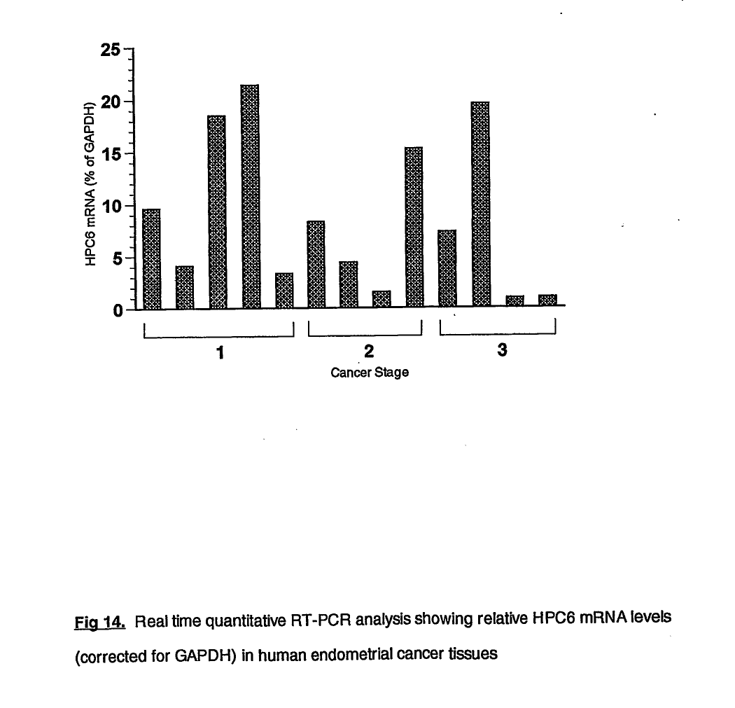

On the DDPCR gel, band 9 was much more intense in implantation sites compared to interimplantation sites in all individual animals tested as shown in FIG. 1A. To verify that this band indeed represents gene(s) which are differentially expressed between the two sites, the cDNA products of band 9 were extracted out of the DDPCR gel, re-amplified and cloned into the pGEM-T vector; Northern blot analysis was performed using the cloned inserts as probes. Among the clones analysed, cDNA of clone 9.5 explicitly detected differential expression of mRNA between the two sites on the Northern blot, with much higher mRNA level present in implantation sites compared to interimplantation sites. This is illustrated in FIG. 1B. On this initial Northern blot, more than one transcript were observed, with a 3.5 kb band being the prominent transcript. This confirmed that clone 9.5 contained the cDNA representing the original expression pattern of band 9 on the DDPCR gel.

### EXAMPLE 5

### Sequence Analysis of Clone 9.5 and Comparison with the Genbank Database

Band 9 resulted from the DDPCR amplification of day 4.5 implantation site mRNA with the following two primers: 5′ primer, GTGACGTAGG (OPA-8, Table 1) and 3′ primer, T12MA (Table 1). After the confirmation that clone 9.5 contained the cDNA representing band 9, the nucleotide sequence of this clone was determined, and is shown in Table 2. It contained 158 nucleotides, and the ends of the sequence indeed contained the unique and expected primer sequence of GTGACGTAGG at the 5′ end, and the reverse complementary sequence of T12MA at the 3′ end (underlined in Table 2). This confirmed that the cDNA in clone 9.5 was indeed the direct PCR product amplified from the specific primers applied during DDPCR amplification.

TABLE 2

The sequence of clone 9.5 (158 bp) derived from band 9 of
DDPCR analysis

| | | |
|---|---|---|
| 1 | GTGACGTAGG ACAGATCGGT GCACTGAAGG ACTACTATCA CTTCTACCAT | SEQ ID NO: 46 |
| 51 | AGTAGGACCA TTAAAAGGTC TGTTCTCTCG AGCAGAGGAA CCCACAGTTT | |
| 101 | CATTTCAATG GAACCAAAGG TGGAGTGGAT CCAACAGCAA GTGGTGAAAA | |
| 151 | AAAAAAAA | |

* The underlined parts represent the primers used during DDPCR amplification

When compared to the GenBank database, the sequence of clone 9.5 showed 98% identity to mouse serine protease PC6 (MUSPC6)(Accession: D12619; 2994 bp), 98% identity to mouse convertase PC5 (MUSPC5)(Accession: L14932; 2848 bp) and 98% identity to human protease PC6 isoform A (HPC6A)(Accession: U56387; 2844 bp). It appears that in fact MUSPC5 and MUSPC6 are actually the same gene under different names, and that they are 90% homologous to HPC6A cDNA. The detailed comparison of clone 9.5 (SEQ ID NOS: 47 and 48) with MUSPC6 cDNA (SEQ ID NOS: 49 and 50) is illustrated in Table 3, showing that clone 9.5 aligned to nucleotide (nt) 254-411 of MUSPC6 cDNA. In principle, and in most experimentally-tested cases, a DDPCR-derived fragment should represent the 3′ end sequence for a cDNA (Liang & Pardee, 1992; Nie et al. 2000); however, in contrast to this expectation, clone 9.5 aligns with the nucleotide sequence 254-411, which is near the 5′ end, but not to the 3′ end of MUSPC6 cDNA (Table 3). This unusual outcome is thought to result from the unique sequence of MUSPC6 cDNA (Table 3) and the intrinsic nature of PCR, in which the 5′ end of a primer sequence can be degenerate.

TABLE 3

The comparison of nucleotide sequence of clone 9.5
(158 bp) with mouse PC6 cDNA

| | |
|---|---|
| | 10 20 30 |
| clone9.5 | GTGACGTAGGACAGATCGGTGCACTGAAGGACT |
| | ::::::::::::::::::::::::::::::::::: |
| MUSPC6 | ATCGCATAGCCAGCAAGTACGGATTCATCAACGTAGGACAGATCGGTGCACTGAAGGACT |
| | 230 240 250 260 270 280 |
| | 100 110 120 130 140 150 |
| clone9.5 | ACAGTTTCATTTCAATGGAACCAAAGGTGGAGTGGATCCAACAGCAAGTGGTGAAAAAAA |
| | :::::::::::::::::::::::::::::::::::::::::::::::::::::::::::: |
| MUSPC6 | ACAGTTTCATTTCAATGGAACCAAAGGTGGAGTGGATCCAACAGCAAGTGGTGAAAAAAA |
| | 350 360 370 380 390 400 |
| CLONE9.5 | AAAAA |
| | :: |
| MUSPC6 | GAACC |
| | 411 |

### EXAMPLE 6

### RT-PCR Cloning of Mouse PC6 (MUSPC6) and Determination of MUSPC6 mRNA Expression in the Uterus During Early Pregnancy

The results shown in FIG. 1B and Table 3 indicated that clone 9.5 represents part of MUSPC6 cDNA, and that the level of expression of this gene is much higher in implantation sites compared to the interimplantation sites on day 4.5 of pregnancy. To confirm the identity of clone 9.5 and verify that MUSPC6 mRNA is indeed differentially expressed between the two sites, two additional cDNA fragments of MUSPC6, designated MUSPC6-fragA and MUSPC6-fragB, were cloned using the RT-PCR approach, and used as cDNA probes on the Northern blots. MUSPC6-fragA was a 171 bp fragment covering nucleotides 4-174, and MUSPC6-fragB was a 422 bp fragment covering nucleotides 501-922 of MUSPC6 cDNA (Accession: D12619; 2994 bp). These two fragments were designed to represent the up- and down-stream region of MUSPC6 cDNA, to which clone 9.5 is aligned. Using MUSPC6-fragA and MUSPC6-fragB as probes, a pattern of mRNA expression similar to that shown in FIG. 1B, where clone 9.5 was used as a probe, was observed between implantation sites and interimplantation sites (data not shown). This conclusively confirmed the identity of clone 9.5 as representing MUSPC6, and showed that MUSPC6 mRNA is up-regulated in implantation sites on day 4.5 of pregnancy.

In a subsequent experiment, MUSPC6-fragB was used as the cDNA probe, and additional Northern analyses were performed to determine the expression pattern of this gene in the uterus in relation to the time of implantation and early pregnancy. Total RNA from the uterus of non-pregnant mice (estrus) and pregnant mice at the initial stage for implantation (day 4.5 of pregnancy) through to fully established implantation and placentation (day 10.5 of pregnancy) was analysed. The results are shown in FIG. 2. Very low expression was observed in non-pregnant mice, as well as in mice on day 3.5 of pregnancy. However, around day 4.5 up to day 6.5 of pregnancy a dramatic upregulation of this gene occurred in the implantation sites, but not in the interimplantation sites (FIG. 2). Beyond day 6.5 of pregnancy, the expression level then drastically decreased, and returned to the level in non-pregnant uterus.

Thus this upregulation was very transient, and only occurred between day 4.5 and 6.5 of pregnancy, when the embryos are actively implanting into the uterus. The upregulation was very implantation site-specific, and did not occur uniformly along the uterine horns, but only in the implantation sites.

In addition, it was noticeable that more bands were observed in FIG. 2 than in FIG. 1B. This was simply due to the different lengths of film exposure for the two Northern blots. After the experiment reported in FIG. 2, the result of FIG. 1B was repeated, and similar numbers of bands were detected after much longer film exposure. Thus it is clear that in implantation sites during the active embryo implantation period, the uterus expresses multiple transcripts of MUSPC6, with a size range of 2.2 kb, 3.5 kb, 6.5 kb and 10 kb; of these the 3.5 kb transcript is the most abundant. This observation agrees with the finding that multiple transcripts were detected for MUSPC6 in the brain and intestine (Nakagawa et al. 1993 a & b).

### EXAMPLE 7

### MUSPC6 mRNA Level During the Estrous Cycle

The influence of the estrous cycle on the expression of MUSPC6 in the non-pregnant uterus was examined by determining the level of MUSPC6 mRNA by Northern analysis. The study utilised 16 individual mice at different stages of the cycle (metestrus, diestrus, proestrus and estrus), grouped to represent 4 cycles, and results from one typical cycle are shown in FIG. 3. In all cases, the expression level was very low at estrus and proestrus and became marginally higher during metestrus and diestrus; however, the levels at all stages of the estrous cycle were much lower than that in the implantation sites during the implantation period. These results indicated that the upregulation observed in implantation sites is not solely a direct result of the influence of the ovarian hormones during pregnancy, but requires other factors, such as the presence of an embryo.

### EXAMPLE 8

### Effects of the Embryo on Uterine Expression of MUSPC6

To determine whether the presence of an embryo in the uterus was essential for the observed changes in MUSPC6 mRNA expression during early pregnancy, total RNA was isolated from mice on day 4.5 of pseudopregnancy, and the mRNA expression pattern compared with that in normal day 4.5 pregnant animals by Northern analysis. The results are shown in FIG. 4. This experiment demonstrated that the mRNA level in the pseudopregnant mice was actually equivalent to that in interimplantation sites of pregnant animals, and was much lower than that at the implantation sites. This implied that the observed increase in MUSPC6 mRNA expression at the implantation sites during early pregnancy required the presence of embryos in the uterus.

### EXAMPLE 9

### Tissue Distribution of MUSPC6 mRNA

Multi-tissue Northern analysis was performed to investigate the tissue distribution of MUSPC6 mRNA expression. The results, illustrated in FIG. 5, show that MUSPC6 is not widely expressed. When an equal amount of total RNA was compared, the implantation sites on day 5.5 showed the highest level of expression, and apart from the uterus, only intestine, ovary, testis and brain among the 12 tissues tested showed detectable expression.

### EXAMPLE 10

### Evidence from a Uterine- and Pregnancy-Specific Form of MUSPC6

MUSPC6 has been previously studied in the brain and intestine (Nakagawa et al. 1993 a & b); therefore intestine was chosen as the positive control tissue for our studies. However, it was observed that the profile of the mRNA transcripts detected by Northern blotting was different in the uterus from that in the intestine. Both tissues show multiple transcripts ranging from 2.2 kb to 10 kb, but there is a subtle difference; the 6.5 kb transcript present in the intestine is replaced by a 5.5 kb one in the uterus, as shown in FIG. 2. This 6.5 kb intestinal transcript has been previously investigated in detail, and shown to encode a MUSPC6 isoform, designated as mouse PC6 B (Accession: D17583, 5208 bp), which has an extremely large cysteine-rich motif (Nakagawa et al., 1993a). This isoform of MUSPC6 (MUSPC6B) is membrane-bound, whereas the previously documented MUSPC6 is soluble (De Bie et al., 1996). Consequently, in order to avoid confusion, the previously documented isoform of MUSPC6 disclosed herein is designated MUSPC6A, and the membrane-bound isoform is designated MUSPC6B. From comparison of the cDNA and protein sequences, it appears that MUSPC6A and MUSPC6B are generated via alternative splicing of the same primary transcript (Nakagawa et al., 1993a). It appears that the 5.5 kb mRNA transcript which we have identified in the uterus is unique to the pregnant uterus, and may encode an isoform of PC6 which is different from PC6A or PC6B.

In order to confirm the presence of a unique 5.5 kb transcript in the implantation sites in the uterus, and to determine whether this 5.5 kb transcript is different from the 6.5 kb transcript present in the intestine and therefore may code for another isoform of MUSPC6, the following approaches were used.

Firstly, cDNA fragments coding for the different domains of mouse MUSPC6 protein were RT-PCR cloned, and these fragments were used as cDNA probes for Northern blotting performed on mouse uterine tissues and the intestine. At the protein level, the MUSPC6 protein consists of the following five domains (in the order from the N- to C-terminal): signal peptide, propeptide, subtilisin-like catalytic domain, homo B domain and cysteine (Cys)-rich domain (Nakagawa et al. 1993). The first four domains are exactly the same between the Sciences, 20th Edition, Williams and Wilkins, Pennsylvania, USA.

The compounds and compositions of the invention may be administered by any suitable route, and the person skilled in the art will readily be able to determine the most suitable route and dose for the condition to be treated. Dosage will be at the discretion of the attendant physician or veterinarian, and will depend on the nature and state of the condition to be treated, the age and general state of health of the subject to be treated, the route of administration, and any previous treatment which may have been administered.

The carrier or diluent, and other excipients, will depend on the route of administration, and again the person skilled in the art will readily be able to determine the most suitable formulation for each particular case.

As used herein, the term “therapeutically effective amount” means an amount of a compound of the present invention effective to yield a desired therapeutic response, for example to prevent or treat a disease which is susceptible to treatment by administration of a pharmaceutically-active agent.

The specific “therapeutically effective amount” will of course vary with such factors as the particular condition being treated, the physical condition and clinical history of the subject, the type of animal being treated, the duration of the treatment, the nature of concurrent therapy (if any), and the specific formulations employed and the structure of the compound or its derivatives.

As used herein, a “pharmaceutical carrier” is a pharmaceutically acceptable solvent, suspending agent, excipient or vehicle for delivering the compound of formula I and/or pharmaceutically-active agent to the subject. The carrier may be liquid or solid, and is MUSPC6A and MUSPC6B isoforms, and differences are found only in the Cys-rich domain, which is at the C-terminus. There are two unique features of the B isoform:
(1) It has a very long Cys-rich domain, which is about four times large as that of the A isoform,
(2) It contains a stretch of hydrophobic amino acids as a putative trans-membrane domain near the C-terminus (Nakagawa et al. 1993).

Based on this protein domain information, the following cDNA fragments were cloned using RT-PCR:
(1) MUSPC6-fragA, 171 bp, covering nt 4-174 of MUSPC6A cDNA (Accession: D12619; 2994 bp), representing the 5′-untranslated region (UTR) and the signal peptide domain;
(2) MUSPC6-fragDA, 177 bp, covering nt 267-443 of MUSPC6A cDNA, representing the propeptide domain;
(3) MUSPC6-fragDB, 479 bp, covering nt 444-922 of MUSPC6A cDNA, representing the subtilisin-like catalytic domain;
(4) MUSPC6-fragG, 653 bp, covering nt 1305-1957 of MUSPC6A cDNA, representing the Homo-B domain;
(5) MUSPC6-fragH, 672 bp, covering nt 2106-2777 of MUSPC6A cDNA, representing the common Cys-rich domain present in both the A and B isoforms;
(6) MUSPC6-fragI, 227 bp, covering nt 2735-2961 of MUSPC6A cDNA, representing the C-terminus and 3′ UTR of the A isoform, and thus representing a cDNA fragment specific to the A isoform only;
(7) MUSPC6-fragK, 377 bp, covering nt 2522-2898 of MUSPC6B cDNA (Accession: D17583, 5208 bp), representing the unique Cys-rich domain of the B isoform;
(8) MUSPC6-fragM, 398 bp, covering nt 4285-4682 of MUSPC6B cDNA, representing the unique trans-membrane domain of the B isoform.

Northern blots containing mouse uterine tissues of non-pregnant (estrus), implantation and interimplantation sites on day 4.5 and 5.5 of pregnant mice and intestine were prepared and probed with the cDNA fragments described above. The results are shown in FIG. 6. When the fragments MUSPC6-fragA/DA/DB/G/H were used as probes, all of the multiple bands observed previously for the uterus and intestine were detected in both type of tissues, and the 6.5 kb B-isoform specific transcript was detected only in the intestine, as shown in FIG. 6A. In all cases, a 5.5 kb uterus-specific transcript was indeed present in the implantation sites, and this transcript seems to have replaced the 6.5 kb transcript present in the intestine, as shown in FIG. 6A.

This indicates that the cDNA sequences tested so far are common to all of the multiple transcripts, and that therefore the protein domains represented by these cDNAs are common to all of the possible isoforms of the MUSPC6 protein. These results also confirmed that the mRNA transcript profiles are different between the uterus and the intestine. When MUSPC6-fragI, an A-isoform specific cDNA fragment, was used as a probe on the same blot, only the 3.5 kb transcript, but not the other transcripts, was detected in all of the tissues; certainly this probe did not detect the 6.5 kb transcript of the intestine, as shown in FIG. 6B, indicating that only the 3.5 kb transcript but not the other ones contains this A-isoform specific cDNA sequence. This result also confirms the known difference between the A and B isoforms at the cDNA level. When the two B-isoform specific cDNA fragments, MUSPC6-fragK and MUSPC6-fragM, were used on the same blot, in both cases the 6.5 kb transcript was detected only in the intestine; no other bands were observed in either the intestine or the uterus, as shown in FIG. 6C.

When these B-isoform specific probes were tested on the multi-organ Northern blot, only the intestine showed a clear 6.5 kb band; other tissues, including muscle, brain and uterus were negative. These results are shown in FIG. 7, and both confirmed the specificity of the probes used in this study, and verified that the B-isoform is only expressed in the intestine, and not in the uterus or other organs.

Collectively, our results confirm that:
(a) the 5.5 kb transcript detected in the uterus is different from the 6.5 kb one in the intestine,
(b) this 5.5 kb transcript is indeed specific to the implantation sites of the uterus during the embryo implantation period, and
(c) this uterine-specific transcript may code for an isoform of MUSPC6 which is different from the previously identified A and from the B isoforms, the difference is mainly at the C-terminal end.

### EXAMPLE 11

### Genomic Southern Analysis of MUSPC6 Gene

Genomic DNA was isolated from mouse uterus and kidney, and the DNA was subjected to Southern analysis. Similar results were obtained for the two tissue types; thus only the results obtained with the uterus will be discussed. FIG. 8 shows the results of Southern analysis of mouse genomic DNA digested separately with BamHI, EcoRI, HindIII and Bg1II and probed with radio-labeled MUSPC6-fragH. In all cases, the digestion pattern was quite simple, and indicate that the MUSPC6 gene is represented by a single copy in the genome.

### EXAMPLE 12

### In Situ Hybridisation of MUSPC6 mRNA in the Uterus of Mice During Early Pregnancy

The cell types which express MUSPC6 mRNA in the uterus were identified by in situ hybridisation, using riboprobes generated against clone 9.5. The sequences of the riboprobes are set out in Table 2. In non-pregnant and in day 3.5 pregnant uterus, no single cell type was distinctively positive. Positive signals were only detected from day 4.5-6.5 of pregnancy at the implantation sites, and they were predominantly localised in the decidualized stromal cells at the anti-mesometrial pole of the uterus, as shown in FIG. 9; throughout this period of time, the interimplantation sites were always negative. Interestingly, at the implantation sites the mesometrial side of the uterus on the same section always had many fewer positive cells, compared to the anti-mesometrial pole. The intensity of the signals was high on day 4.5 and 5.5, and then decreased dramatically on day 6.5. After day 6.5 of pregnancy, no signals were detected in any cells in the uterus.

These results indicate that MUSPC6 mRNA is only expressed in the decidual zones of the uterus around the embryo implantation period, and that this expression is quite transient, and specific to the decidua. The expression is not uniformly distributed at the implantation sites; the anti-mesometrial pole, where the embryo contacts with the uterus during implantation, showed higher expression, and the opposite side, the mesometrial pole, showed a much lower level of expression. Interestingly, some of the cells in artificially decidualized uterus were also positive for MUSPC6 probe. This indicates that MUSPC6 is involved in the decidualization process, and that it may not be restricted to the embryo-induced decidualization, but also occurs in other decidualization events which can be induced by other means, such as oil.

### EXAMPLE 13

### Detection of PC6 in Human Uterus and Other Human Tissues

A human PC6 cDNA probe was prepared by RT-PCR and cloned, and applied to a human multi-tissue array (Clontech). The results are illustrated in FIG. 10. A strong positive signal was detected in the heart, gastrointestinal tract, kidney, thymus, lung, placenta, uterus, testis, ovary, in similar fetal organs, and in colorectal adenocarcinoma.

A human multiorgan Northern blot with 1(g of polyA+ RNA (Clontech), applied to each lane (was probed as shown in FIG. 11. Strong positive signals were obtained for heart, kidney, small intestine, placenta, and lung. Transcript sizes detected were around 10 kb, 6.5 kb and 3.5 kb.

FIG. 12 shows the results of semi-quantitative RT-PCR Southern blot analysis of PC6 in human endometrium taken across the menstrual cycle. Primers used were upper primer: 5′ GAT CGG TGC ACT GAA GGA CTA 3′ (SEQ ID NO: 35), lower primer: 5′ CCA GCA TTC GCA CTC CTC 3′ (SEQ ID NO: 51). An expected band of 545 bp was detected in all samples.

FIG. 13 shows the results of similar semiquantitative RT-PCR analysis in first trimester decidua and placenta, heart, pre- and post-menopausal ovary and term placenta. Thus it is apparent that PC6 is expressed in the human endometrium and a number of other human tissues. Its expression in decidua and placenta of early pregnancy suggests a role in human pregnancy.

### EXAMPLE 14

### Expression of PC6 in Human Endometrial Cancers

FIG. 14 shows the results of real-time quantitative RT-PCR performed using primers on 13 samples of endometrial cancer. Messenger RNA values for PC 5/6 have been corrected for loading, as assessed by quantitation for glyceraldehyde phosphate dehydrogenase. PC6 was detected in every cancer sample, and for each grade of the cancer (1-3), International Federation of Gynaecology and obstetrics Classification).

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed on the following pages, and are incorporated herein by this reference.

### REFERENCES

Abrahamsohn P A and Zorn T M T Implantation and decidualization in rodents. J Exp Zool 1993; 266: 603-628.
Bayer E A, Wilchek M. Protein biotinylation. Methods Enzymol. 1990;184:138-60.
Boudreault A, Gauthier D, Lazure C. Proprotein convertase PC1/3-related peptides are potent slow tight-binding inhibitors of murine PC1/3 and Hfurin. J. Biol. Chem. 1998 Nov. 20;273 (47):31574-80.
Brodeur et al., In: Monoclonal Antibody Production Techniques and Applications, Marcel Dekker, Inc., New York, 1987, pp. 51-63.
Bruggermann et al., Year in Immunology 1993; 7:33.
Chomczynski P and Sacchi N Single-step method of RNA isolation by acid guanidinium thiocyanate-phenol-chloroform extraction. Anal. Biochem. 1987; 162: 156-159.
Das S K, Lim H, Paria B C et al. Cyclin D3 in the mouse uterus is associated with the decidualization process during early pregnancy. J. Mol. Endocrinol. 1999; 22: 91-101.
David G S, Reisfeld R A. Protein iodination with solid state lactoperoxidase. Biochemistry. 1974 Feb. 26;13(5):1014-21.
De Bie, I., Marcinkiewicz, M., Malide, D. et al. The isoforms of proprotein convertsas PC5 are sorted to different subcellular compartments. J. Cell Biol. 1996; 135:1261-1275.
Finn, C. A. and McLaren, A. A study of the early stages of implantation in mice. J Reprod Fert. 1967; 13: 259-267.
Finn, C. A. and Pope, M. Vascular and Cellular changes in the decidualized endometrium of the ovariectomized mouse following cessation of hormone treatment: a possible model for menstruation. J. Endrocrinol. 1984; 100: 295-300.
Hoogenboom H R, Winter G. By-passing immunisation. Human antibodies from synthetic repertoires of germline VH gene segments rearranged in vitro. J. Mol. Biol. 1992 Sep. 20;227(2):381-8.
Huet-Hudson Y M, Chakraborty C, De S K et al. Estrogen regulates the synthesis of epidermal growth factor in mouse uterine epithelial cells. Mol. Endocrinol. 1990; 4: 510-523.
Jakobovits A, Moore A L, Green L L et al. Germ-line transmission and expression of a human-derived yeast artificial chromosome. Nature. 1993b Mar. 18;362(6417):255-8.
Jakobovits A, Vergara G J, Kennedy J L et al., Analysis of homozygous mutant chimeric mice: deletion of the immunoglobulin heavy-chain joining region blocks B-cell development and antibody production. Proc Natl Acad Sci USA. 1993a Mar. 15;90(6):2551-5.
Jean F, Stella K, Thomas L, Liu G, Xiang Y, Reason A J, Thomas G. Alpha1-Antitrypsin Portland, a bioengineered serpin highly selective for furin: application as an antipathogenic agent. Proc Natl Acad Sci USA. 1998 Jun. 23;95(13):7293-8.
Jones P T, Dear P H, Foote J et al., Replacing the complementarity-determining regions in a human antibody with those from a mouse. Nature. 1986 May 29-Jun. 4;321(6069):522-5.
Jones R W and Jones M J Simplified filter paper sandwich blot provides rapid, background-free Northern blots. BioTechniques. 1992; 12: 685-688.
Kohler G, Milstein C. Continuous cultures of fused cells secreting antibody of predefined specificity. Nature. 1975 Aug. 7;256(5517):495-7.
Komminoth P Digoxigenin as an alternative probe labeling for in situ hybridization. Diagn Mol Pathol 1992; 1: 142-150.
Kozbor D, Tripputi P, Roder J C et al., A human hybrid myeloma for production of human monoclonal antibodies. J. Immunol. 1984 December;133(6):3001-5.
Langer R, Brem H, Tapper D. Biocompatibility of polymeric delivery systems for macromolecules. J Biomed Mater Res. 1981 March;15(2):267-77.
Langer et al., Chem. Tech. 1982; 12:98-105.
Liang P and Pardee A B Differential display of eukaryotic messenger RNA by means of the polymerase chain reaction. Science 1992; 257: 967-970.
Liang P and Pardee A B Distribution and cloning of eukaryotic mRNAs by means of differential display: refinement and optimization. Nucleic Acids Res. 1993; 14: 3269-3275.
Marks J D, Hoogenboom H R, Bonnert T P et al., By-passing immunization. Human antibodies from V-gene libraries displayed on phage. J. Mol. Biol. 1991 Dec. 5;222(3):581-97.
Miranda L, Wolf J, Pichuantes S, Duke R, Franzusoff A. Isolation of the human PCG gene encoding the putative host protease for HIV-1 gp160 processing in CD4+ T lymphocytes. Proc Natl Acad Sci USA. 1996 Jul. 23;93(15):7695-700.
Morrison S L, Johnson M J, Herzenberg L A et al., Chimeric human antibody molecules: mouse antigen-binding domains with human constant region domains. Proc Natl Acad Sci USA. 1984 November;81(21):6851-5.
Nakagawa, T., Murakami, K. and Nakayama, K. Identification of an isoform with an extremely large Cys-rich region of PC6, a kex2-like processing endoprotease. FEBS. 1993a; 327: 165-171.
Nakagawa, T., Hosaka, M., Torii, S. et al. Identification and functional expression of a new member of the mammalian Kex2-like processing endoprotease family: its striking structural similarity to PACE4. J. Biochem. 1993b; 113:132-135.
Nie G-Y, Butt A R, Salamenson L A et al. Hormonal and non-hormonal agents at implantation as targets for contraception. Reprod Fertil Dev 1997; 9: 65-76.
Nie G-Y, Li Y, Wang J et al. Complex regulation of calcium-binding protein D9k (Calbindin-D9k) in the mouse uterus during early pregnancy and at the site of embryo implantation. Biol Reprod. 2000a; 62: 27-36.
Nie G-Y, Li Y, Hampton A L et al. Identification of monoclonal nonspecific suppressor factor beta (MNSFbeta) as one of the genes differentially expressed at implantation sites compared to interimplantation sites in the mouse uterus. Mol Reprod Dev. 2000b; 55: 351-363.
Pain D, Surolia A. Preparation of protein A-peroxidase monoconjugate using a heterobifunctional reagent, and its use in enzyme immunoassays. J Immunol Methods. 1981;40(2):219-30.
Psychoyos A In: Handbook of Physiology, vol II (Female Reprodctive System), part 2., American Physiological Society, Washington, 1973, pp. 187-215.
Rancourt, S. L. and Rancourt, D. E. Murine subtilisin-like proteinase SPC6 is expressed during embryonic implantation, somitogenesis, and skeletal formation. Deve. Genet. 1997; 21:75-81.
Riechmann L, Clark M, Waldmann H et al., Reshaping human antibodies for therapy. Nature. 1988 Mar. 24;332(6162):323-7.
Robb L, Li R, Hartley L et al. Infertility in female mice lacking the receptor for interleukin 11 is due to a defective uterine response to implantation. Nature Medicine 1998; 4: 303-308.
Rugh, R. The mouse. Its reproduction and development. 1994. New York, Oxford University Press.
Sambrook, J., Fritsch, E. F., and Maniatis, T. Molecular cloning: a laboratory manual. 1989. Cold Spring Harbor, N.Y., Cold Spring Harbor Laboratory Press.
Seidah, N. and Chretien, M. Proprotein and prohormone convertases: a family of subtilases generating diverse bioactive polypeptides. Brain Research. 1999; 848:45-62.
Sidman K R, Steber W D, Schwope A D et al., Controlled release of macromolecules and pharmaceuticals from synthetic polypeptides based on glutamic acid. Biopolymers. 1983 January;22(1):547-56.
Stewart C L, Kaspar P, Brunet L J et al. Blastocyst implantation depends on maternal expression of leukaemia inhibitory factor. Nature 1992; 359: 76-79.
Verhoeyen M, Milstein C, Winter G. Reshaping human antibodies: grafting an antilysozyme activity. Science. 1988 Mar. 25;239(4847):1534-6.
Zhong M, Scott Munzer S, Basak A, Benjannet S, Mowla S J, decroly E, Chreitien M, Seidah N G. The prosegments of furin and PC7 as potent inhibitors of proprotein convertases. J. Biol. Chem. 1999; 274:33913-33920.
Zhu L-J, Cullinan-Bove K, Polihronis M et al. Calcitonin is a progesterone-regulated marker that forecasts the receptive state of endometrium during implantation. Endocrinology 1998; 139: 3923-3934.
Zola Monoclonal Antibodies: A Manual of Techniques, CRC Press Inc, 1987; pp. 147-158.

1-29. (canceled)

30. A nucleic acid molecule encoding an isoform of the protein proprotein convertase (PC) 5/6, which nucleic acid molecule:
(a) encodes a uterus-specific RNA transcript of about 5.5 kb;
(b) is expressed during the implantation period at implantation sites in a pregnant uterus but is not detectably expressed in intestine or other tissues where PC 5/6 protein may be present;
(c) under at least moderately stringent conditions, is able to hybridize to DNA of sequence SEQ ID NO:4 but not to DNA of sequence SEQ ID NO:5 and SEQ ID NO:6.

31. A nucleic acid molecule according to claim 30 which is in the anti-sense orientation.

32. A nucleic acid molecule according to claim 30, which is a cDNA molecule, a genomic DNA molecule, or an RNA molecule.

33. A nucleic acid molecule according to claim 32, wherein the nucleic acid molecule is a cDNA molecule.

34. A nucleic acid molecule according to claim 30, which:
(a) comprises the sequence SEQ. ID. NO: 1 or SEQ ID NO:2; or
(b) is able to hybridize under at least moderately stringent conditions to DNA having the sequence SEQ ID NO:1 or SEQ ID NO:2; or
(c) has at least 75% sequence identity to SEQ ID NO:1 or SEQ ID NO: 2.

35. A nucleic acid molecule according to claim 34, which is able to hybridize under stringent conditions to DNA with the sequence SEQ. ID. NO:1 or SEQ ID NO: 2.

36. A nucleic acid molecule according to claim 34, which has at least 80%, sequence identity to SEQ ID NO:1 or SEQ ID NO:2.

37. An isolated protein having PC 5/6 activity which is encoded by the nucleic acid molecule of claim 30.

38. A pharmaceutical composition comprising the PC 5/6 protein of claim 37, together with a pharmaceutically acceptable carrier.

39. A method of promoting fertility in a female mammalian subject in need of such fertility promotion, comprising the step of stimulating the activity of proprotein convertase (PC) 5/6 in the uterus of a said subject.

40. A method according to claim 39, wherein the activity is stimulated by administering a PC 5/6 polypeptide, or an agonist of PC 5/6.

41. A method according to claim 39, wherein the PC 5/6 is an isoform of the form of PC 5/6 that is specifically expressed in implantation sites of the uterus during embryo implantation.

42. A method according to claim 39, wherein the uterus of the subject is converted from a non-receptive state to a receptive state.

43. A method according to claim 39, wherein the stimulating promotes implantation of a fertilized egg in the uterus.

44. A method according to claim 39, wherein the subject suffers from a fertility-related condition.

45. A method according to claim 44, wherein the fertility-related condition is luteal phase defect, failure of implantation, pre-eclampsia, early abortion, intrauterine growth restriction, abnormal uterine bleeding, endometriosis or early parturition.

46. A method according to claim 39, wherein the subject is to be implanted with one of her own fertilized eggs.

47. A method according to claim 39, wherein the subject is to be implanted with a donor's fertilized egg.

48. A method of promoting fertility in a female mammalian subject in need of such fertility promotion, comprising the step of stimulating, in the uterus of a said subject, the activity of a PC 5/6 isoform that is encoded by the nucleic acid molecule of claim 19.

49. A method of detecting whether a female mammalian subject is in a fertile state or period, whether the uterus of said subject is in a receptive state, or whether the subject is in an early stage of pregnancy, comprising
obtaining a first biological sample from the subject and detecting the presence or measuring the activity of PC 5/6 in said sample, and, optionally, comparing the result to a second or greater biological sample (i) obtained during a nonfertile period, or (ii) obtained at a stage of the estrous cycle when the uterus is not receptive, or (iii) obtained from a nonpregnant subject, wherein:
(a) the presence of PC 5/6, or an increase in the activity of PC 5/6 in the first sample relative to the activity in the second sample obtained during a nonfertile period, indicates that the subject is in a fertile state or period;
(b) the presence of PC 5/6 or an increased amount of PC 5/6 in the first sample compared with the PC 5/6 from the second sample from a nonreceptive stage of the cycle indicates that the uterus is in the receptive state; and
(c) the presence of PC 5/6 activity in the first sample or an increase of PC 5/6 in said first sample compared to the level in the second sample from a non-pregnant subject indicates said early stage of pregnancy.

50. A method according to claim 49, wherein the biological sample is uterine tissue, washings from a uterine cavity, blood, plasma, serum or saliva.

51. A method of inhibiting fertility in a female mammalian subject in need or desirous thereof, comprising the step of administering an antagonist of PC 5/6 to said subject.

52. A method according to claim 51, wherein the antagonist inhibits conversion of a uterine non-receptive state to a receptive state.

53. A method according to claim 51, wherein the antagonist is an anti-PC 5/6 antibody or an anti-sense nucleic acid that specifically hybridizes in vivo with a nucleic acid encoding PC 5/6.

54. A method according to claim 51, wherein the antagonist inhibits embryo implantation.

55. A method of (i) screening a sample or collection of compounds for a compound that modulates the enzymatic activity of PC 5/6 protein, (ii) testing a candidate compound for said modulating activity, or (iii) testing a candidate compound for PC 5/6 enzymatic activity, the method comprising:
(a) providing a composition comprising said PC 5/6 protein and assessing the ability of said sample or collection, or said candidate compound, to increase or decrease the enzymatic activity of said protein relative to the activity of said PC 5/6 protein in the absence of said sample, collection or candidate compound; or
(b) testing the candidate compound for it ability to catalyze the conversion of protein precursors into mature proteins, which protein precursors are ones that are cleaved by PC 5/6 to become mature proteins.


SEQUENCE LISTING (51 sequences)

SEQ ID NO: 1 - DNA, 2994 bp, Mus sp.
gaagttagttgtgcgcgctgcttagcgcgcgagccagcgggcgggcgaaggcggcgaagcgtcgggaccatggactgggactgggggaaccgctgcagccgcccgggacggcgggacctgctgtgcgtgctggcactgctcgccggctgtctgctcccggtatgccggacgcgcgtctacaccaaccactgggcagtgaagatcgccggcggcttcgcggaggcagatcgcatagccagcaagtacggattcatcaacgtaggacagatcggtgcactgaaggactactatcacttctaccatagtaggaccattaaaaggtctgttctctcgagcagaggaacccacagtttcatttcaatggaaccaaaggtggagtggatccaacagcaagtggtgaaaaaaagaaccaagagggattatgacctcagccatgcccagtcaacctacttcaatgatcccaagtggccaagtatgtggtacatgcactgcagtgacaacacccatccttgccagtcagacatgaatatcgaaggagcctggaagagaggctacacggggaagaatatcgtggtgactatcctggatgacggcatcgagagaacccacccagatctgatgcaaaactacgatgctctggcaagttgcgatgtgaatgggaatgacttggacccgatgcctcgttatgatgcaagcaatgagaacaagcatgggacccgctgtgccggagaagtggcagccactgcaaacaactctcactgcactgtcgggatcgctttcaacgccaagattggaggtgtgagaatgctggatggtgatgtcactgacatggtggaggcaaagtctgtcagctacaacccacagcatgtgcacatctacagtgccagctggggaccagatgacgatggcaagactgtggatgggccagctcccctcacccggcaagcctttgagaatggcgtgagaatggggcggagaggccttggatctgtgtttgtgtgggcatctggcaatggtggacggagcaaggatcactgttcttgtgatggctataccaacagcatctataccatctccatcagcagtacggccgaaagtggaaagaaaccttggtacttggaagagtgttcatctacactggctacaacctacagcagtggagaatcctatgataagaaaataatcactactgatctaaggcagcgatgcacagacaatcacactggaacgtcagcctcagcccccatggctgctggaatcattgccctggccctagaagccaatccgtttctgacctggagagacgtgcagcatgttattgtcaggacttcccgtgcgggacatttgaacgctaatgactggaaaaccaatgctgctggttttaaggtgagccatctctatggatttggactgatggatgccgaagccatggtgatggaagcagagaagtggacaactgttcctcagcagcacgtgtgtgtggaaagcacagaccgacaaatcaagaccattcgaccaaacagtgcagtgcgctccatctacaaagcctcaggctgctcggataatcccaaccatcacgtcaattacctggagcatgtagttgtgcgtattaccatcacacacccacggaggggagacctggccatctatctgacatcaccctcaggaaccagatcccagctcttggccaacaggctctttgatcattccatggaagggtttaagaactgggagttcatgactattcattgctggggagaacgggctgctggggactgggtcctcgaagtttatgatacgccatctcagctgaggaacttcaagactccaggtaaattgaaagaatggtccttagtcctctatggcacgtccgtacagccatactccccaaccaacgagtttcccaaagtggaacgcttccgctacagccgagtggaagaccccacagatgactacggtgctgaagattatgcaggtccctgtgaccctgaatgcagtgaggttggatgtgacgggccaggaccagatcactgcagtgactgcttacactactactacaagctgaaaaataacaccagaatctgtgtctccagctgccctcctggccactaccatgctgacaagaagaggtgccggaagtgtgccccaaactgcgagtcctgctttggcagccatggtgatcagtgcctctcctgtaaatatggctacttcctgaatgaagaaactagcagctgtgttactcagtgccctgatggatcatacgaggatatcaagaaaaatgtctgtgggaaatgcagtgagaactgcaaggcatgcattggatttcacaactgcacagagtgcaagggcgggttaagtcttcagggatcccgctgttcggtcacctgcgaggatggacagttcttcaatggtcacgactgccagccctgccatcgcttctgtgctacttgctctggggccggagcagatggatgtattaactgcaccgaggggtatgtcatggaagagggaaggtgtgtacaaagttgtagtgtgagctactacttggaccactcttcagagggtggctacaaatcctgcaagagatgtgataacagctgtttgacatgcaatgggccaggattcaagaactgttccagctgccccagtggatatcttttagacttaggaacgtgtcagatgggagccatctgcaaggatgctacggaagagtcctgggcagaaggaggcttctgtatgcttgtgaaaaagaacaatctctgtcagcgaaaagttctacaacagctgtgctgcaaaacatgtacattccaaggctgagcagccgccttccatccctgttcctgtagacgtatagattattccatattattaaagaaaaaaaaagccaaaaaactgcaagccacctctctctttcttgctatcttttttttctctctctctctatctcaagtttgtgcagggagatggtgaactgttttgtcagaacttaaatag

SEQ ID NO: 2 - DNA, 5208 bp, Mus sp.
aacagcatctataccatctccatcagcagtacggccgaaagtggaaagaaaccttggtacttggaagagtgttcatctacactggctacaacctacagcagtggagaatcctatgataagaaaataatcactactgatctaaggcagcgatgcacagacaatcacactggaacgtcagcctcagcccccatggctgctggaatcattgccctggccctagaagccaatccgtttctgacctggagagacgtgcagcatgttattgtcaggacttcccgtgcgggacatttgaacgctaatgactggaaaaccaatgctgctggttttaaggtgagccatctctatggatttggactgatggatgccgaagccatggtgatggaagcagagaagtggacaactgttcctcagcagcacgtgtgtgtggaaagcacagaccgacaaatcaagaccattcgaccaaacagtgcagtgcgctccatctacaaagcctcaggctgctcggataatcccaaccatcacgtcaattacctggagcatgtagttgtgcgtattaccatcacacacccacggaggggagacctggccatctatctgacatcaccctcaggaaccagatcccagctcttggccaacaggctctttgatcattccatggaagggtttaagaactgggagttcatgactattcattgctggggagaacgggctgctggggactgggtcctcgaagtttatgatacgccatctcagctgaggaacttcaagactccaggtaaattgaaagaatggtccttagtcctctatggcacgtccgtacagccatactccccaaccaacgagtttcccaaagtggaacgcttccgctacagccgagtggaagaccccacagatgactacggtgctgaagattatgcaggtccctgtgaccctgaatgcagtgaggttggatgtgacgggccaggaccagatcactgcagtgactgcttacactactactacaagctgaaaaataacaccagaatctgtgtctccagctgccctcctggccactaccatgctgacaagaagaggtgccggaagtgtgccccaaactgcgagtcctgctttggcagccatggtgatcagtgcctctcctgtaaatatggctacttcctgaatgaagaaactagcagctgtgttactcagtgccctgatggatcatacgaggatatcaagaaaaatgtctgtgggaaatgcagtgagaactgcaaggcatgcattggatttcacaactgcacagagtgcaagggcgggttaagtcttcagggatcccgctgttcggtcacctgcgaggatggacagttcttcaatggtcacgactgccagccctgccatcgcttctgtgctacttgctctggggccggagcagatggatgtattaactgcaccgaggggtatgtcatggaagagggaaggtgtgtacaaagttgtagtgtgagctactacttggaccactcttcagagggtggctacaaatcctgcaagagatgtgataacagctgtttgacatgcaatgggccaggattcaagaactgttccagctgccccagtggatatcttttagacttaggaacgtgtcagatgggagccatctgcaaggatggagaatacattgatgaccaaggccactgccaaacctgtgaagcctcatgtgccaagtgctggggaccaactcaggaagactgtatcagctgccccgtaacaagggtcttggatgatggtcgctgtgttatgaactgtccttcctggaagttcgaatttaagaagcaatgccatccctgccactacacttgtcaaggatgccaaggtagtggcccttccaactgcacctcctgtagagcagacaagcatggtcaggagcgcttcctgtaccacggggaatgtctggagaactgccctgtgggccattatcctgccaagggacacacctgcctaccctgcccagacaactgtgagctttgctacaacccacacatctgcagtcgatgcatgagtggctatgtcatcattccccccaaccacacctgccagaagctggagtgcagacaaggtgaattccaggattctgagtatgaagaatgcatgccctgtgaggaaggatgtctgggatgcactgaggatgatccaggagcctgtacctcgtgtgctacaggatattacatgtttgagcggcattgctataaagcctgtcctgagaagaccttcggtgtgaaatgggaatgcagggcctgtggtactaactgtggcagctgcgaccaacatgagtgctactggtgtgaggagggcttctttctctccggtggcagctgtgtgcaggattgtggccctggcttccatggagaccaagagttgggagaatgcaagccctgccaccgagcctgtgagacttgcactggctcgggctacaaccaatgcagcagctgtcaagaagggttgcagctatggcatgggacgtgtctctggtccacctggcctcaggtggaaggcaaggactggaatgaagccgtgcccactgaaaagccatctttggtgaggagtctgctgcaggatcgacgaaagtggaaagttcaaatcaaaagagatgcaacgagccagaatcaaccttgtcactcttcttgtaaaacctgcaatggatctctctgcgcttcatgtcccacaggtatgtacctgtggctgcaggcctgtgttccttcctgtccccaaggcacttggccatcagtcaccagtggcagctgtgaaaagtgttccgaggactgtgtctcctgctccggtgccgacctttgccaacagtgcctgagccagccggacaacactctgcttctccatgagggcaggtgctaccacagttgcccagagggcttttatgcaaaagatggtgtttgtgaacactgtagttccccttgcaaaacatgcgaaggaaatgccaccagctgtaactcttgtgaaggagacttcgtcctagaccatggggtgtgctggaaaacttgccctgaaaagcacgtggccgtggaaggagtctgcaagcactgtccagagaggtgccaggactgcatccacgagaaaacttgcaaagagtgcatgcctgacttctttctatacaatgacatgtgccatcgttcctgtcccaagagcttctaccctgacatgcgccagtgtgtcccctgccacaaaaactgtctggagtgcaatggccccaaggaagatgactgtaaggtctgtgctgatacttctaaggctctccacaatggattgtgcttggatgagtgccctgagggaacctacaaagaagaagagaatgatgaatgcagagattgccccgagtcctgcctgatctgctcatcagcttggacctgcctggcctgtcgggaaggcttcacagtagtgcatgatgtctgcacggcacccaaggagtgtgcagccgtcgagtactgggatgagggctcccacaggtgccagccgtgtcacaagaaatgctcccgctgctcagggccttctgaggaccagtgctatacctgtcccagagagacttttcttctcaatacaacctgtgtgaaagagtgcccagagggctaccacactgacaaggacagccagcagtgtgtgctctgccacagctcttgcaggacctgcgaaggaccacacagcatgcagtgcctctcctgccgacctggctggtttcaactgggcaaggagtgcttgctgcaatgcagggatggatattatggagaaagcaccagcggtaggtgtgagaagtgtgacaagagctgcaagtcatgccgaggtccacggcccacagactgccagtcttgcgatacatttttctttctcctacgctccaagggacagtgccaccgcgcttgccctgagcactactatgcagaccaacacgcccagacctgtgagaggtgccaccccacttgtgacaagtgcagcggaaaggaggcgtggagttgcttgtcctgtgtgtggagctaccaccttctgaaaggaatctgcatcccagaatgtatcgtaggggaatacagagagggaaagggagaaaactttaactgtaaaaaatgccacgagagctgcatggaatgcaagggtccaggcagcaagaactgcaccgggtgctcagctggcctgctgctggacatggacgacaaccgctgcctccattgctgcaatgcctcccactcccgcagatcccaggactgctgcgactgccagagttccacagatgagtgcatccttccagccagggaggctgagttttacgagcacaccaagactgctctgctagtgacctctggcgccatgctgttgctgctgctgggggctgctgcggtcgtgtggcggaagtctcgaagcagacctgtggcaaaggggcggtacgaaaagctggcagaacctaccgtgtcatactcctcctacaggagcagctatcttgacgaggaccaggtgattgagtacagggaccgggactacgatgaggacgatgaggacgacatcgtctatatgggccaagatggcactgtctaccggaagttcaagtatgggctgctggatgagacggaagatgatgagttggagtacgatgatgagagctactcttaccaataaacagagccccctcccatctcaaacccaccacccaccacccacctcccatcccttcctggtcccttcctggctctcaatcctgctgggagctaccaatgcattggctgaggtctgagtgtttgcttttgtccttaccttgcatccaacccgaatatgtagggatgctggagtctgctgtgctgcttttcattagctgagtgcaatgaacagatcctgcaaggagagaattcgaaggcattttccccgagggtatgtcaagggtattaaatgcaggaatctaaaagaactaagatctgttgaacactctaatgtgatttttaaagtggatggagaagaggacctgtaagggttctgctttcaaactgtagttggagactcactctcatccccacaactgctaccttctagaattctgatatttgactaaatagaggggagatggagtactagagagttgttggagctggggtgggaggtctctccactttcaatcgtggaaagaaagaaagaaagaaagaaagaaagaaagaaagaaagaaagaaagaaagaaagaaagaaag

SEQ ID NO: 3 - DNA, 2766 bp, Homo sapiens
agcgtcgggaccatggattgggattgggggaaccgctgcagccgcccgggacggcgggacctgctgtgcgtgctggcactgctcgccggctgtctgctcccggtatgccggacgcgcgtctacaccaaccactgggcagtgaagatcgccggcggcttcgcggaggcagatcgcatagccagcaagtacggattcatcaacgtaggacagatcggtgcactgaaggactactatcacttctaccatagtaggaccattaaaaggtctgttctctcgagcagaggaacccacagtttcatttcaatggaaccaaaggtggagtggatccaacagcaagtggtgaaaaaaagaaccaagagggattatgacctcagccatgcccagtcaacctacttcaatgatcccaagtggccaagtatgtggtacatgcactgtagcgacaatacacatccctgccagtctgacatgaatatcgaaggagcctggaagagaggctacacgggaaagaacattgtggtcactatcctggatgacggaattgagagaacccatccagatctgatgcaaaactacgatgctctggcaagttgcgacgtgaatgggaatgacttggacccaatgcctcgttatgatgcaagcaacgagaacaagcatgggactcgctgtgctggagaagtggcagccgctgcaaacaattcgcactgcacagtcggaattgctttcaacgccaagatcggaggagtgcgaatgctggacggagatgtcacggacatggttgaagcaaaatcagttagcttcaacccccagcacgtgcacatttacagcgccagctggggcccggatgatgatggcaagactgtggacggaccagcccccctcacccggcaagcctttgaaaacggcgttagaatggggcggagaggcctcggctctgtgtttgtttgggcatctggaaatggtggaaggagcaaagaccactgctcctgtgatggctacaccaacagcatctacaccatctccatcagcagcactgcagaaagcggaaagaaaccttggtacctggaagagtgttcatccacgctggccacaacctacagcagcggggagtcctacgataagaaaatcatcactacagatctgaggcagcgttgcacggacaaccacactgggacgtcagcctcagcccccatggctgcaggcatcattgcgctggccctggaagccaatccgtttctgacctggagagacgtacagcatgttattgtcaggacttcccgtgcgggacatttgaacgctaatgactggaaaaccaatgctgctggttttaaggtgagccatctttatggatttggactgatggacgcagaagccatggtgatggaggcagagaagtggaccaccgttccccggcagcacgtgtgtgtggagagcacagaccgacaaatcaagacaatccgccctaacagtgcagtgcgctccatctacaaagcttcaggctgctcggataaccccaaccgccatgtcaactacctggagcacgtcgttgtgcgcatcaccatcacccaccccaggagaggagacctggccatctacctgacctcgccctctggaactaggtctcagcttttggccaacaggctatttgatcactccatggaaggattcaaaaactgggagttcatgaccattcattgctggggagaaagagctgctggtgactgggtccttgaagtttatgatactccctctcagctaaggaactttaagactccaggtaaattgaaagaatggtctttggtcctctacggcacctccgtgcggccatattcaccaaccaatgaatttccgaaagtggaacggttccgctatagccgagttgaagaccccacagacgactatggcacagaggattatgcaggtccctgcgaccctgagtgcagtgaggttggctgtgacgggccaggaccagaccactgcaatgactgtttgcactactactacaagctgaaaaacaataccaggatctgtgtctccagctgcccccctggccactaccacgccgacaagaagcgctgcaggaagtgtgcccccaactgtgagtcctgctttgggagccatggtgaccaatgcatgtcctgcaaatatggatactttctgaatgaagaaaccaacagctgtgttactcactgccctgatgggtcatatcaggataccaagaaaaatctttgccggaaatgcagtgaaaactgcaagacatgtactgaattccataactgtacagaatgtagggatgggttaagcctgcagggatcccggtgctctgtctcctgtgaagatggacggtatttcaacggccaggactgccagccctgccaccgcttctgcgccacttgtgctggggcaggagctgatgggtgcattaactgcacagagggctacttcatggaggatgggagatgcgtgcagagctgtagtatcagctattactttgaccactcttcagagaatggatacaaatcctgcaaaaaatgtgatatcagttgtttgacgtgcaatggcccaggattcaagaactgtacaagctgccctagtgggtatctcttagacttaggaatgtgtcaaatgggagccatttgcaaggatgcaacggaagagtcctgggcggaaggaggcttctgtatgcttgtgaaaaagaacaatctgtgccaacggaaggttcttcaacaactttgctgcaaaacatgtacattccaaggctgagcagcc

SEQ ID NO: 4 - DNA, 2734 bp, Mus sp.
gaagttagttgtgcgcgctgcttagcgcgcgagccagcgggcgggcgaaggcggcgaagcgtcgggaccatggactgggactgggggaaccgctgcagccgcccgggacggcgggacctgctgtgcgtgctggcactgctcgccggctgtctgctcccggtatgccggacgcgcgtctacaccaaccactgggcagtgaagatcgccggcggcttcgcggaggcagatcgcatagccagcaagtacggattcatcaacgtaggacagatcggtgcactgaaggactactatcacttctaccatagtaggaccattaaaaggtctgttctctcgagcagaggaacccacagtttcatttcaatggaaccaaaggtggagtggatccaacagcaagtggtgaaaaaaagaaccaagagggattatgacctcagccatgcccagtcaacctacttcaatgatcccaagtggccaagtatgtggtacatgcactgcagtgacaacacccatccttgccagtcagacatgaatatcgaaggagcctggaagagaggctacacggggaagaatatcgtggtgactatcctggatgacggcatcgagagaacccacccagatctgatgcaaaactacgatgctctggcaagttgcgatgtgaatgggaatgacttggacccgatgcctcgttatgatgcaagcaatgagaacaagcatgggacccgctgtgccggagaagtggcagccactgcaaacaactctcactgcactgtcgggatcgctttcaacgccaagattggaggtgtgagaatgctggatggtgatgtcactgacatggtggaggcaaagtctgtcagctacaacccacagcatgtgcacatctacagtgccagctggggaccagatgacgatggcaagactgtggatgggccagctcccctcacccggcaagcctttgagaatggcgtgagaatggggcggagaggccttggatctgtgtttgtgtgggcatctggcaatggtggacggagcaaggatcactgttcttgtgatggctataccaacagcatctataccatctccatcagcagtacggccgaaagtggaaagaaaccttggtacttggaagagtgttcatctacactggctacaacctacagcagtggagaatcctatgataagaaaataatcactactgatctaaggcagcgatgcacagacaatcacactggaacgtcagcctcagcccccatggctgctggaatcattgccctggccctagaagccaatccgtttctgacctggagagacgtgcagcatgttattgtcaggacttcccgtgcgggacatttgaacgctaatgactggaaaaccaatgctgctggttttaaggtgagccatctctatggatttggactgatggatgccgaagccatggtgatggaagcagagaagtggacaactgttcctcagcagcacgtgtgtgtggaaagcacagaccgacaaatcaagaccattcgaccaaacagtgcagtgcgctccatctacaaagcctcaggctgctcggataatcccaaccatcacgtcaattacctggagcatgtagttgtgcgtattaccatcacacacccacggaggggagacctggccatctatctgacatcaccctcaggaaccagatcccagctcttggccaacaggctctttgatcattccatggaagggtttaagaactgggagttcatgactattcattgctggggagaacgggctgctggggactgggtcctcgaagtttatgatacgccatctcagctgaggaacttcaagactccaggtaaattgaaagaatggtccttagtcctctatggcacgtccgtacagccatactccccaaccaacgagtttcccaaagtggaacgcttccgctacagccgagtggaagaccccacagatgactacggtgctgaagattatgcaggtccctgtgaccctgaatgcagtgaggttggatgtgacgggccaggaccagatcactgcagtgactgcttacactactactacaagctgaaaaataacaccagaatctgtgtctccagctgccctcctggccactaccatgctgacaagaagaggtgccggaagtgtgccccaaactgcgagtcctgctttggcagccatggtgatcagtgcctctcctgtaaatatggctacttcctgaatgaagaaactagcagctgtgttactcagtgccctgatggatcatacgaggatatcaagaaaaatgtctgtgggaaatgcagtgagaactgcaaggcatgcattggatttcacaactgcacagagtgcaagggcgggttaagtcttcagggatcccgctgttcggtcacctgcgaggatggacagttcttcaatggtcacgactgccagccctgccatcgcttctgtgctacttgctctggggccggagcagatggatgtattaactgcaccgaggggtatgtcatggaagagggaaggtgtgtacaaagttgtagtgtgagctactacttggaccactcttcagagggtggctacaaatcctgcaagagatgtgataacagctgtttgacatgcaatgggccaggattcaagaactgttccagctgccccagtggatatcttttagacttaggaacgtgtcagatgggagccatctgcaaggatgctacggaagagtcctgggcagaaggaggcttct

SEQ ID NO: 5 - DNA, 377 bp, Mus sp.
tggaaggcaaggactggaatgaagccgtgcccactgaaaagccatctttggtgaggagtctgctgcaggatcgacgaaagtggaaagttcaaatcaaaagagatgcaacgagccagaatcaaccttgtcactcttcttgtaaaacctgcaatggatctctctgcgcttcatgtcccacaggtatgtacctgtggctgcaggcctgtgttccttcctgtccccaaggcacttggccatcagtcaccagtggcagctgtgaaaagtgttccgaggactgtgtctcctgctccggtgccgacctttgccaacagtgcctgagccagccggacaacactctgcttctccatgagggcaggtgctaccacagttgcccagag

SEQ ID NO: 6 - DNA, 398 bp, Mus sp.
agggaggctgagttttacgagcacaccaagactgctctgctagtgacctctggcgccatgctgttgctgctgctgggggctgctgcggtcgtgtggcggaagtctcgaagcagacctgtggcaaaggggcggtacgaaaagctggcagaacctaccgtgtcatactcctcctacaggagcagctatcttgacgaggaccaggtgattgagtacagggaccgggactacgatgaggacgatgaggacgacatcgtctatatgggccaagatggcactgtctaccggaagttcaagtatgggctgctggatgagacggaagatgatgagttggagtacgatgatgagagctactcttaccaataaacagagccccctcccatctcaaacccaccacccac

SEQ ID NO: 7 - DNA, 14 bp, Artificial Sequence
  Synthetic nucleotide - primer
ttttttttttttva

SEQ ID NO: 8 - DNA, 14 bp, Artificial Sequence
  Synthetic nucleotide - primer
ttttttttttttvc

SEQ ID NO: 9 - DNA, 14 bp, Artificial Sequence
  Synthetic nucleotide - primer
ttttttttttttvg

SEQ ID NO: 10 - DNA, 14 bp, Artificial Sequence
  Synthetic nucleotide - primer
ttttttttttttvt

SEQ ID NO: 11 - DNA, 10 bp, Artificial Sequence
  Synthetic nucleotide  - primer
caggcccttc

SEQ ID NO: 12 - DNA, 10 bp, Artificial Sequence
  Synthetic nucleotide  - primer
tgccgagctg

SEQ ID NO: 13 - DNA, 10 bp, Artificial Sequence
  Synthetic nucleotide - primer
agtcagccac

SEQ ID NO: 14 - DNA, 10 bp, Artificial Sequence
  Synthetic nucleotide primer
aatcgggctg

SEQ ID NO: 15 - DNA, 10 bp, Artificial Sequence
  Synthetic nucleotide - primer
aggggtcttg

SEQ ID NO: 16 - DNA, 10 bp, Artificial Sequence
  Synthetic nucleotide - primer
ggtccctgac

SEQ ID NO: 17 - DNA, 10 bp, Artificial Sequence
  Synthetic nucleotide - primer
gaaacgggtg

SEQ ID NO: 18 - DNA, 10 bp, Artificial Sequence
  Synthetic nucleotide - primer
gtgacgtagg

SEQ ID NO: 19 - DNA, 10 bp, Artificial Sequence
  Synthetic nucleotide - primer
gggtaacgcc

SEQ ID NO: 20 - DNA, 10 bp, Artificial Sequence
  Synthetic nucleotide -primer
gtgatcgcag

SEQ ID NO: 21 - DNA, 10 bp, Artificial Sequence
  Synthetic nucleotide - primer
caatcgccgt

SEQ ID NO: 22 - DNA, 10 bp, Artificial Sequence
  Synthetic nucleotide - primer
tcggcgatag

SEQ ID NO: 23 - DNA, 10 bp, Artificial Sequence
  Synthetic nucleotide - primer
cagcacccac

SEQ ID NO: 24 - DNA, 10 bp, Artificial Sequence
  Synthetic nucleotide - primer
tctgtgctgg

SEQ ID NO: 25 - DNA, 10 bp, Artificial Sequence
  Synthetic nucleotide - primer
ttccgaaccc

SEQ ID NO: 26 - DNA, 10 bp, Artificial Sequence
  Synthetic nucleotide - primer
agccagcgaa

SEQ ID NO: 27 - DNA, 10 bp, Artificial Sequence
  Synthetic nucleotide - primer
gaccgcttgt

SEQ ID NO: 28 - DNA, 10 bp, Artificial Sequence
  Synthetic nucleotide  - primer
aggtgaccgt

SEQ ID NO: 29 - DNA, 10 bp, Artificial Sequence
  Synthetic nucleotide - primer
caaacgtcgg

SEQ ID NO: 30 - DNA, 10 bp, Artificial Sequence
  Synthetic nucleotide - primer
gttgcgatcc

SEQ ID NO: 31 - DNA, 21 bp, Artificial Sequence
  Synthetic nucleotide - primer
gttagttgtgcgcgctgctta

SEQ ID NO: 32 - DNA, 16 bp, Artificial Sequence
  Synthetic nucleotide - primer
gcgcgtccggcatacc

SEQ ID NO: 33 - DNA, 20 bp, Artificial Sequence
  primer
cacccatccttgccagtcag

SEQ ID NO: 34 - DNA, 21 bp, Artificial Sequence
  Synthetic nucleotide - primer
catccacagtcttgccatcgt

SEQ ID NO: 35 - DNA, 21 bp, Artificial Sequence
  primer
gatcggtgcactgaaggacta

SEQ ID NO: 36 - DNA, 20 bp, Artificial Sequence
  Synthetic nucleotide - primer
ctggcagcatgttattgtca

SEQ ID NO: 37 - DNA, 21 bp, Artificial Sequence
  Synthetic nucleotide  - primer
cgtagtcatctgtggggtctt

SEQ ID NO: 38 - DNA, 20 bp, Artificial Sequence
  Synthetic nucleotide - primer
ccactaccatgctgacaaga

SEQ ID NO: 39 - DNA, 21 bp, Artificial Sequence
  Synthetic nucleotide - primer
agaacttttcgctgacagaga

SEQ ID NO: 40 - DNA, 21 bp, Artificial Sequence
  Synthetic nucleotide - primer
gtatgcttgtgaaaaagaaca

SEQ ID NO: 41 - DNA, 21 bp, Artificial Sequence
  Synthetic nucleotide - primer
ccctgcacaaacttgagatag

SEQ ID NO: 42 - DNA, 21 bp, Artificial Sequence
  Synthetic nucleotide - primer
tggaaggcaaggactggaatg

SEQ ID NO: 43 - DNA, 21 bp, Artificial Sequence
  Synthetic nucleotide - primer
ctctgggcaacttgtgtagca

SEQ ID NO: 44 - DNA, 21 bp, Artificial Sequence
  Synthetic nucleotide - primer
agggaggctgagttttacgag

SEQ ID NO: 45 - DNA, 18 bp, Artificial Sequence
  Synthetic nucleotide - primer
gtgggtggtgggtttgag

SEQ ID NO: 46 - DNA, 158 bp, Mus sp.
  Synthetic nucleotide - primer
gtgacgtaggacagatcggtgcactgaaggactactatcacttctaccatagtaggaccattaaaaggtctgttctctcgagcagaggaacccacagtttcatttcaatggaaccaaaggtggagtggatccaacagcaagtggtgaaaaaaaaaaaa

SEQ ID NO: 47 - DNA, 33 bp, Artificial
  Synthetic nucleotide - primer
gtgacgtaggacagatcggtgcactgaaggact

SEQ ID NO: 48 - DNA, 65 bp, Artificial
  Synthetic nucleotide - primer
acagtttcatttcaatggaaccaaaggtggagtggatccaacagcaagtggtgaaaaaaaaaaaa

SEQ ID NO: 49 - DNA, 60 bp, Mus sp.
atcgcatagccagcaagtacggattcatcaacgtaggacagatcggtgcactgaaggact

SEQ ID NO: 50 - DNA, 65 bp, Mus sp.
acagtttcatttcaatggaaccaaaggtggagtggatccaacagcaagtggtgaaaaaaagaacc

SEQ ID NO: 51 - DNA, 18 bp, Artificial sequence
  Synthetic nucleotide - primer
ccagcattcgcactcctc